(19) United States
(12) Patent Application Publication
(10) Pub. No.: US 2004/0180394 A1
(43) Pub. Date: Sep. 16, 2004

(54) Assays for inositol phosphates

(76) Inventors: Philip E. Brandish, Blue Bell, PA (US); Lorraine A Hill, Jim Thorpe, PA (US)

Correspondence Address: MERCK AND CO INC, P O BOX 2000, RAHWAY, NJ, 070650907

(21) Appl. No.: 10/483,572

(22) PCT Filed: Jul. 17, 2002
(86) PCT No.: PCT/US02/23379

(30) Foreign Application Priority Data
Jul. 20, 2001 (US) 60/306732

(51) Int. Cl. C12Q 1/42

(52) U.S. Cl. 435/21

(57) ABSTRACT

The present invention provides cell-based assays for inositol phosphates involving the preferential binding of radio-labeled inositol phosphates to a solid phase containing a scintillant within. The assay allows one to screen for inhibitors of inositol phosphate phosphatases or GPCRs which are coupled to phosphoinositide hydrolysis. The assays are fast, convenient, and avoid the column chromatography steps that prior art methods employed.

### CROSS-REFERENCE TO RELATED APPLICATIONS

Not applicable.

### STATEMENT REGARDING FEDERALLY-SPONSORED R&D

Not applicable.

### REFERENCE TO MICROFICHE APPENDIX

Not applicable.

### FIELD OF THE INVENTION

The present invention is directed to methods of measuring inositol phosphates, especially where the inositol phosphates are found in cells and the methods are used to provide an assay for the activity of receptor proteins that are coupled to the inositol phosphate pathway.

### BACKGROUND OF THE INVENTION

G-protein coupled receptors (GPCRs) are a very large class of membrane receptors that relay information from the exterior of cells to the interior. GPCRs function by interacting with a class of heterotrimeric proteins known as G-proteins. Most GPCRs function by a similar mechanism. Upon the binding of agonist, a GPCR catalyzes the dissociation of guanosine diphosphate (GDP) from the a subunit of G proteins. This allows for the binding of guanosine triphosphate (GTP) to the a subunit, resulting in the disassociation of the a subunit from the β and γ subunits. The freed a subunit then interacts with other cellular components, and in the process passes on the extracellular signal represented by the presence of the agonist. Occasionally, it is the freed β and γ subunits which transduce the agonist signal.

Ligand binding to certain G-protein coupled receptors (GPCRs), followed by the release of G-protein subunits, results in the activation of phospholipase C. Phospholipase C catalyzes the hydrolysis of phosphatidylinositol 4,5-bisphosphate (PIP₂) to diacylglycerol and an inositol phosphate, myo-inositol 1,4,5-triphosphate (IP₃). Diacylglycerol activates protein kinase C and IP₃ mobilizes intracellular calcium, leading to the production of a diverse array of intracellular messengers (Fisher et al., 1984, Trends Biochem. Sci. 9:53-58; Berridge et al., 1983, Biochem. J. 212:473-482; Agranoffet al., 1983, J. Biol. Chem. 258:2076-2078; Nishizuka, 1984, Science 225:1365-1370; Kaibuche et al., 1983, J. Biol. Chem. 258:6701-6704). For publications discussing inositol phosphates and their role in cellular signaling, see, e.g., Irvine & Schell, 2001, Nature Rev. 2:327-328; Shears, 2000, Bioessays 9:786-789; Majerus et al., 1999, J. Biol. Chem. 274:10669-10672; Shears, 1998, Biochim. Biophys. Acta 1436:49-67; Acharya et al., 1998, Neuron 20:1219-1229; Balla, 2001, Current Pharmaceutical Design 7:475-507.

GPCR activity is often monitored by the measurement of changes in intracellular inositol phosphate levels. This is generally done by labeling cells containing the GPCR with [³H]-myo-inositol, activating the GPCR with agonist, and then adding formic acid to terminate the production of inositol phosphates. Often, the cells are exposed to lithium chloride (LiCl) during the activation step. Treatment of cells with LiCl prevents breakdown of inositol phosphates to inositol. Under this condition, the mass of soluble inositol phosphate is a quantitative measure of GPCR activation. This is not the case if the inositol phosphates are degraded to inositol because inositol is used to resynthesize the phosphoinositide lipid which is the precursor for the inositol phosphates. Thus, treatment with LiCl makes the assay quantitative, in addition to boosting the signal. After the reaction has been terminated, cellular extracts are prepared and the level of inositol phosphates in the extracts is measured. This requires separating inositol phosphates from inositol, usually by the use of large volumes of ion exchange resins, a labor intensive and time consuming process. For examples of such methods, see Tian et al., 1997, J. Biomol. Screening 2, 91-97; Wriggett & Irvine, 1987, Biochem. J. 245:655-660; Davidson et al., 1990, Endocrinol. 126:80-87; Horwitz & Perlman, 1987, Meth. Enzymol. 141:169-175; Berridge et al., 1982, Biochem. J. 206:587-595; Huckle & Conn, 1987, Meth. Enzymol. 141:141-155.

The level of inositol phosphates in a cell is determined primarily by the balance between synthetic pathways such as those mediated by GPCRs and degradative pathways. Degradative pathways are the result of the activity of a variety of enzymes. For example, IP₃ is degraded by a series of inositol phosphatases to inositol monophosphate (IP₁). Inositol polyphosphate 5-phosphatase converts I(1,4,5)P₃ into I(1,4)P₂. I(1,4)P₂ is converted into inositol 4-monophosphate by (I(4)P₁) by inositol polyphosphate 1-phosphatase. Finally, I(4)P₁ is dephosphorylated by inositol monophosphatase which can then be re-incorporated into phosphatidylinositol.

Inositol monophosphatase is inhibited by lithium. Therefore, it is common to include lithium chloride in assays to determine the levels of inositol phosphates so that the cycle outlined above is stopped at the level of IP₁. Measuring the level of IP₁ can then serve as a surrogate for measuring the level of all the inositol phosphates and IP₁ can be measured without the complication of having to take into account the reincorporation of IP₁ into phosphatidylinositol.

The measurement of inositol phosphates usually entails labeling cells with [³H]-myo-inositol and following the radioactive label into ³H-IP₁. At some point in this process, neutral [³H]-myo-inositol is separated from charged ³H-IP₁. Prior art measurements of inositol phosphates required time consuming and labor intensive ion exchange chromatography steps to make this separation.

Inositol phosphatases are a class of enzymes that remove phosphate groups from inositol phosphates and participate in certain signal transduction pathways. One kind of inositol phosphatase is represented by the inositol polyphosphate 5-phosphatase family. This family of enzymes removes the 5 phosphate from inositol- and phosphatidylinositol-polyphosphates. Members of this family are identified by their substrate specificity and amino acid sequence homology to one another. See Jefferson & Majerus, 1995, J. Biol. Chem. 270:9370-9377.

Tian et al., 1997, J. Biomol. Screening 2:91-97 described an assay for receptor-mediated phosphatidylinositol turnover that employed anion exchange columns that were prepared directly on fiber glass 96-well multiscreen microtiter filter plates.

Chengalvala et al., 1999, J. Biochem. Biophys. Methods 38:163-170 described an assay for quantitation of inositol phosphates in biological samples that utilized 96-well microtiter plates that had been fitted with filtration disks containing regenerated Dowex AG1-X8 resin.

### SUMMARY OF THE INVENTION

The present invention is directed to a cell-based assay for inositol phosphates involving the preferential binding of radiolabeled inositol phosphates to a solid phase containing a scintillant within. The assay allows one to screen for inhibitors of inositol phosphate phosphatases or to monitor the activity of cellular proteins such as certain GPCRs which are coupled to phosphoinositide hydrolysis. The assay does not include the cumbersome chromatography steps that are part of prior art inositol phosphate assays. Thus, the assay is fast, easy to apply, and lends itself well to automation for high throughput.

### BRIEF DESCRIPTION OF THE DRAWINGS

FIG. 1A shows the general characteristics of a bead suitable for use in the present invention. The bead contains a positive charge on its outer surface and contains a scintillant within. A preferred example of such beads are the yttrium silicate (YSi) scintillation proximity assay (SPA) beads sold by Amersham Pharmacia Biotech (catalog number RPNQ0013). These beads are underivatized YSi glass beads that have scintillant properties by virtue of cerium ions within the crystal lattice of the beads.

FIG. 1B shows that the YSi SPA beads sold by Amersham Pharmacia Biotech can be used to detect ³H-inositol phosphate (ins-1p) more efficiently than ³H-inositol (inositol). See Example 1 for details.

FIG. 2A shows that non-radioactive inositol-1-phosphate, but not inositol, competes with ³H-inositol-1-phosphate for binding to YSi SPA beads. ³H-inositol-1-phosphate was mixed with YSi SPA beads and counted as described in Example 1 except that the indicated concentrations of either unlabeled inositol-1-phosphate or unlabeled inositol were added to the mixture of beads and ³H-inositol-1-phosphate.

FIG. 2B shows that the YSi SPA beads efficiently detect ³H-inositol-1-phosphate in the presence of 20 mM or 100 mM formic acid. ³H-inositol-1-phosphate or ³H-inositol was mixed with YSi SPA beads and counted as described in Example 1 except that formic acid was added to a final concentration of 20 mM or 100 mM to the bead/³H-inositol-1-phosphate or bead/³H-inositol mixture in the wells of a microtiter plate. The plate was agitated for 1 hr on a plate shaker at room temperature. The beads were allowed to settle for 2 hr at room temperature and were then counted in a Topcount Instrument (Packard Instruments).

FIG. 3 shows a schematic outline of the steps of an embodiment of the present invention. The freeze/thaw steps are optional.

FIG. 4A shows the results of an embodiment of the present invention that is an assay that detects activation of the M1-muscarinic acetylcholine receptor using 20 μl of cell lysate. See Example 3 for details.

FIG. 4B shows the results of an embodiment of the present invention that is an assay that detects activation of the M1-muscarinic acetylcholine receptor using 100 μl of cell lysate. See Example 3 for details.

FIG. 4C shows the results of the control samples for the experiments shown in FIG. 4A and FIG. 4B. See Example 3 for details.

FIG. 4D shows the results when experiments similar to those in FIG. 4A are run in varying concentrations of either LiCl or NaCl.

FIG. 4E shows an experiment similar to that in FIG. 4D except that the detection steps used filtration through Millipore filterplates (as in Chengalvala et al., 1999, J. Biochem. Biophys. Methods 38:163-170) rather than YSi SPA beads.

FIGS. 5A-E shows the results of an embodiment of the present invention that is an assay that detects activation of the M1-muscarinic acetylcholine receptor in transfected T24 cells. See Example 9 for details. Each part of the figure consists of four bars. Reading from left to right the four bars represent: control (no LiCl or carbachol); 5 mM LiCl; 1 mM carbachol; 5 mM LiCl plus 1 mM carbachol. FIGS. 5A-C shows the results of positive control experiments using filtration through Millipore filterplates (as in Chengalvala et al., 1999, J. Biochem. Biophys. Methods 38:163-170) rather than YSi SPA beads. FIG. 5A shows the results from the flow-through; FIG. 5B shows the results from the wash; FIG. 5C shows the results from the eluate. FIG. 5D shows the results of practicing the invention using YSi SPA beads. FIG. 5E shows the results of practicing the invention using polylysine SPA beads. In the case of both the YSi SPA beads and the polylysine SPA beads, the assay successfully detected receptor activation when the cells were treated with carbachol alone (third bars from left in FIG. 5D and FIG. 5E). The detection of receptor activation was even more robust in the presence of both carbachol and LiCl (fourth bars from left in FIG. 5D and FIG. 5E).

FIGS. 6A-D shows the results of an embodiment of the present invention that is an assay that detects activation of the human neuropeptide FF receptor in transfected CHO/NFAT cells. See Example 10 for details. Each part of the figure consists of four bars. Reading from left to right the four bars represent: control (no LiCl or NPFF); 5 mM LiCl; 10 nM NPFF; 5 mM LiCl plus 10 nM NPFF.

FIGS. 6A-C shows the results of positive control experiments using filtration through Millipore filterplates (as in Chengalvala et al., 1999, J. Biochem. Biophys. Methods 38:163-170) rather than YSi SPA beads. FIG. 6A shows the results from the flow-through; FIG. 6B shows the results from the wash; FIG. 6C shows the results from the eluate. FIG. 6D shows the results of practicing the invention using YSi SPA beads. The invention gives results that are essentially equivalent to the prior art method but with a much simpler, much faster set of steps.

FIGS. 7A-D shows the results of negative control experiments that were done as in FIGS. 6A-D except that the cells used were untransfected CHO/NFAT cells, i.e., cells that did not express the human neuropeptide FF receptor.

FIG. 8 shows that poly-L-lysine beads can be used in the methods of the present invention. See Example 11 for details.

FIG. 9 shows that the invention can be used to detect carbachol, an agonist of acetylcholine receptors, which are naturally expressed in HEK293 cells. This demonstrates that the invention can be used to identify agonists of receptors that are naturally expressed in cells. See Example 8 for details.

### DETAILED DESCRIPTION OF THE INVENTION

For the purposes of this invention:

Unless the context indicates otherwise, “inositol phosphates” refers to the entire family of inositol phosphates, e.g., myo-inositol 1,4,5-triphosphate (1(1,4,5)P₃); myo-inositol 1,3,4-triphosphate (1(1,3,4)P₃); myo-inositol 4,5-diphosphate (IP₂); and myo-inositol monophosphate (IP₁).

“Substances” can be any substances that are generally screened in the pharmaceutical industry during the drug development process. For example, substances may be low molecular weight organic compounds (e.g., having a molecular weight of less than about 1,000 daltons), RNA, DNA, antibodies, peptides, or proteins.

The conditions under which cells are incubated with or exposed to substances in the methods described herein are conditions that are typically used in the art for the study of protein-ligand interactions: e.g., physiological pH; salt conditions such as those represented by such commonly used buffers as PBS or in tissue culture media; a temperature of about 4° C. to about 55° C.; incubation times of from several seconds to several hours. Generally, the cells are present in the wells of a multiwell tissue culture plate such as a microtiter plate and the substances are added directly to the wells, optionally after first washing away the media in the wells.

The present invention is directed to a method that is a cell-based assay for inositol phosphates. In its broadest version, the invention is directed to a method of measuring inositol phosphates in cells that comprises: preparing a lysate from cells in which inositol phosphates have been radiolabeled, mixing the lysate with a solid phase that is a material that contains positive charges on its surface and a scintillant within so that the radiolabeled inositol phosphates in the lysate adhere to the solid phase and activate the scintillant, and measuring the amount of scintillation from the solid phase. The assay can be used as part of a method to screen for inhibitors of inositol phosphate (IP) phosphatases or as part of a general method to assay the activity of any G-protein coupled receptor (GPCR) which naturally couples to phosphoinositide hydrolysis or which can be coupled to the phosphoinositide hydrolysis pathway by recombinant techniques such as those described herein.

The present invention is a variety of scintillation proximity assay (SPA). In a SPA, there is a solid phase (e.g., a bead or the bottom of a tissue culture well) that is or contains within it a substance capable of fluorescing when stimulated by a β-particle that has been emitted by a weakly emitting β-isotope such as ³H or ¹²⁵I. The fluorescent substance is known as a scintillant. The surface of the solid phase is such that it has an affinity for the particular analyte the assay is designed to detect. This can be done by modifying the surface of the solid so that it is coated with a receptor where the analyte is a substance that has an affinity for the receptor, e.g., a ligand of the receptor. In the case of the present invention, where the analyte is an inositol phosphate, the surface can be unmodified, provided that the surface of the solid phase carries a positive charge. An example of such an unmodified surface with a positive charge would be that of yttrium silicate. The negative charges of the phosphate groups of inositol phosphates bind to the positive charges on the surface of the yttrium silicate, causing the inositol phosphates to adhere to the surface of the yttrium silicate. Inositol, however, lacking the negatively charged phosphate groups of inositol phosphates, binds to a much lesser extent.

In a SPA, a fluid sample suspected of containing an analyte that has been radiolabeled is brought into contact with the solid phase. If the sample really does contain the analyte, the analyte will bind to the surface of the solid phase. This will bring the analyte into close proximity to the fluorescent substance in the solid phase, such that the radioactive decay products of the radiolabeled analyte will be close enough to interact with the fluorescent substance in the solid phase, stimulating the fluorescent substance to emit light. This emitted light can be detected by suitable means, e.g., with a scintillation counter. Under the proper conditions, the amount of light emitted is proportional to the amount of radiolabeled analyte in the sample. Radioactive decay products or energy emitted by the radioactive analyte have a limited range of travel in the fluid. Therefore, radiolabeled material in the sample that is not the analyte will not bind to the surface of the solid phase and so will be disposed too far away from the fluorescent substance to cause light emission.

In the present invention, the solid phase is generally a material that contains positive charges on its surface and a scintillant within. Alternatively, as in the preferred embodiment discussed below, a glass solid phase is doped with a rare earth element and the doped solid phase itself has scintillating properties.

A preferred example of a solid phase for use in the present invention is cerium-doped yttrium silicate (Y₂SiO₅:Ce). Cerium-doped yttrium silicate is sold as YSi SPA beads by Amersham Pharmacia Biotech (Uppsala, Sweden) as catalog number RPNQ0013. These YSi SPA beads have the following properties:

an average diameter of 2.5 μm

a density of about 4 g/cm³

a settling time of 30-60 minutes in aqueous solutions

a counting window of 5-560 for ³H in a Wallac Microbeta®/Trilux

a counting window of 5-650 for ¹²⁵I in a Wallac Microbeta®/Trilux

a counting window of 0.00-50.00 for ³H in a Packard TopCount®

a counting window of 0.00-100.00 for ¹²⁵I in a Packard TopCount®

YSi SPA beads are generally stored lyophilized, in which state they are stable for 12 months. After reconstitution, the beads should be stored at 2-8° C., preferably in the presence of an anti-bacterial agent such as sodium azide. The exact shelf life of the reconstituted beads will depend somewhat on the reconstitution buffer used. Suitable buffers include: 1% sucrose (w/v); PBS, pH 7.4; Tris, pH 7.4; Hepes, pH 7.4; HBS pH 7.4. These buffers can be supplemented with 0.05% azide (w/v). The composition of these buffers can be found in standard reference texts such as e.g., Sambrook, Fritsch, and Maniatis, 1989, Molecular Cloning: A Laboratory Manual, second edition, Cold Spring Harbor Laboratory Press.

Another preferred solid phase is a glass bead coated with polylysine. An example such a solid phase is the poly-L-lysine SPA beads sold by Amersham Pharmacia Biotech as catalog number RPNQ0010.

A variety of solid phases are suitable for use in the present invention. Because the assays of the present invention are generally performed in aqueous medium, the solid phase should be insoluble in water. The preferred solid phases consist of base glasses which when appropriately activated or doped emit detectable photons of light when excited by the kinetic interaction of nuclear decay particles. Preferably, the activating material or dopant is selected from the groups consisting of: Ce, Mn, Cu, Pb, Sn, Au, Ag, and Sm. The most preferred solid phase is yttrium silicate glass activated with from about 0.1 to about 10.0 percent by weight of an inorganic cerium (Ce) salt. Cerium can be added to the yttrium silicate as an inorganic salt such as the oxide, carbonate, or chloride.

The solid phase is generally formed into beads, i.e., sphere-like particles and can be prepared by methods well known in the art of glass manufacturing. Preferably, the beads have a diameter of from about 1 μm to about 100 μm, more preferably from about 1.5 μm to about 50 μm, even more preferably from about 2 μm to about 10 μm, and most preferably about 2.5 μm. The precise diameter suitable for a particular purpose will depend somewhat on the nature of the radioisotope that is to be detected and selection of the proper diameter is within the knowledge of those skilled in the art.

Alternatively, the solid phase can be prepared by any methods known in the art that result in a solid phase having a positive surface charge and a scintillant within. For example, U.S. Pat. No. 4,568,649 describes such a method wherein the solid phase is soaked in a solvent for the scintillant which is miscible in water in order to dehydrate the solid phase. The solid phase is then placed in a solution composed of the scintillant in the solvent so that the scintillant is integrated into the solid phase. The solid phase containing the scintillant is next placed in an aqueous solution which precipitates the scintillant within the solid phase, thereby locking the scintillant within the solid phase.

In alternative embodiments, the solid phase is a multiwell tissue culture plate in which the walls and/or the bottoms of the wells have been impregnated with a scintillant. The walls and/or bottoms of the wells possess a surface positive charge, or the surfaces of the walls and bottoms of the wells can be coated with a substance having a positive charge. When a cell lysate is added to the wells, inositol phosphates present in the lysate will adhere to the positive charges on the walls and bottoms of the wells while inositol in the lysate will remain in solution in the lysate. If the inositol phosphates and inositol are radiolabeled, only the inositol phosphates (by virtue of adhering to the surfaces of the walls and bottom) will be in close enough proximity to the scintillant to excite it and give rise to a signal.

An example of a suitable multiwell tissue culture plate is the FlashPlate® sold by the NEN® Life Science Products, Inc. The FlashPlate® has a scintillant impregnated into the walls of the plate's wells but the walls have no surface coating. In order to provide a positive charge to the walls, the wells can be treated with an aqueous solution of poly-L-lysine or poly-D-lysine by well-known methods.

The invention involves the measurement of inositol phosphates that have been labeled with a radioisotope. Examples of suitable radioisotopes for use in the present invention are ³H and ¹⁴C. Preferably, the radioisotope is ³H. Electrons emitted by ³H have an average energy of only 6 keV and a very short path length of only about 1 μm in water. Methods of labeling the inositol phosphates with radioisotope are well known in the art. Preferably, cells are grown in medium containing a precursor of the inositol phosphates (e.g., myo-inositol) that has been labeled with the radioisotope. myo-inositol is a precursor of phosphoinositides, which in turn are precursors of inositol phosphates.

The methods of the present invention have various uses. The methods can be used to assay for the activity of inositol phosphate phosphatases such as inositol monophosphatase, inositol polyphosphate 5-phosphatase, or inositol polyphosphate 4-phosphatase. In its broadest version, the invention is directed to a method of measuring inositol phosphates in cells that comprises: preparing a lysate from cells in which inositol phosphates have been radiolabeled, mixing the lysate with a solid phase that is a material that contains positive charges on its surface and a scintillant within so that the radiolabeled inositol phosphates in the lysate adhere to the solid phase and activate the scintillant, and measuring the amount of scintillation from the solid phase.

The present invention provides a method of identifying substances that are inhibitors of inositol phosphate phosphatases. In general terms, the method can be practiced as follows. Test cells are grown or incubated in medium containing no inositol. The medium is then supplemented with inositol that has been labeled with a radioisotope and the test cells are cultured for a period sufficient to permit the uptake of the labeled inositol into the test cells such that a portion of the inositol and inositol phosphates in the test cells becomes labeled. The medium is replaced with fresh medium without inositol, followed by or together with the addition of a substance that is to be tested as a possible inhibitor of inositol phosphate phosphatases. The test cells are incubated with the substance for a period sufficient for the substance to inhibit the inositol phosphate phosphatases in the test cells if the substance is in fact an inhibitor. The medium is removed, the test cells are lysed, and test lysates are prepared. Control lysates are also prepared from control cells that are essentially the same as the test cells and that have been treated in the same manner as the test cells, except that the control cells are not exposed to the substance. Optionally, the test and control cells can be exposed to an agonist for an appropriate GPCR expressed by the cells (e.g., carbochol for the M1-T24 cells disclosed herein) in order to activate phospholipase C. Treatment with the agonist effects hydrolysis of PIP₂ and consequent accumulation of soluble inositol phosphates.

The test and control lysates, containing radiolabeled inositol phosphates and radiolabeled inositol, are brought into contact with an appropriate solid phase such as a scintillation bead with a positive surface charge. The lysates and the solid phase are incubated to allow inositol phosphates in the lysates to bind to the solid phase while inositol remains in the lysate. The scintillation from the solid phase, due to the adhered inositol phosphates, is detected by a suitable instrument. If the substance is an inhibitor of an inositol phosphate phosphatase, the substance will have prevented some of the labeled inositol phosphates in the test cells from being degraded into inositol. Thus, the level of labeled inositol phosphates in the test cells will have been greater than the level of labeled inositol phosphates in the control cells. This will be reflected in the lysates, with the lysate from the test cells having a higher level of labeled inositol phosphates than the lysate from the control cells. Therefore, there will be more radioactivity (i.e., scintillation) detected from the test lysate than from the control lysate if the substance is an inhibitor.

Accordingly, the present invention provides a method of identifying inhibitors of an inositol phosphate phosphatase comprising:

(a) adding inositol that has been labeled with a radioisotope to test cells that express the inositol phosphate phosphatase so that the inositol that has been labeled with a radioisotope is incorporated into inositol phosphates in the test cells;

(b) incubating the test cells with a substance for a period sufficient for the substance to inhibit inositol phosphate phosphatases in the test cells;

(c) lysing the test cells and preparing a test lysate from the test cells;

(d) bringing the test lysate into contact with a solid phase so that inositol phosphates from the test lysate adhere to the solid phase while inositol from the test lysate does not adhere to the solid phase;

(e) determining the amount of radioactivity adhered to the solid phase in step (d);

(f) adding inositol that has been labeled with a radioisotope to control cells that express the inositol phosphate phosphatase so that the inositol that has been labeled with a radioisotope is incorporated into inositol phosphates in the control cells;

(g) incubating the control cells in the absence of the substance for a period essentially the same as the period in step (b);

(h) lysing the control cells and preparing a control lysate from the control cells;

(i) bringing the control lysate into contact with a solid phase so that inositol phosphates from the control lysate adhere to the solid phase while inositol from the control lysate does not adhere to the solid phase;

(j) determining the amount of radioactivity adhered to the solid phase in step (i);

where if the amount of radioactivity determined in step (e) is greater than the amount of radioactivity determined in step (j) then the substance is an inhibitor of the inositol phosphate phosphatase.

The step of determining the amount of radioactivity adhered to the solid phase of steps (e) and (j) can be conveniently carried out by measuring the total amount of radioactivity (e.g., by scintillation counting) in the mixtures of lysates and solid phases since essentially all of the scintillation results from the radioactivity adhered to the beads (i.e., from the inositol phosphates) and very little scintillation results from the radioactivity of the inositol in the solution phase of the lysates.

For the sake of convenience, the steps of adding labeled inositol to the test cells and to the control cells (steps (a) and (f), respectively) can be carried out at the same time. That is, one can label a single population of cells with inositol, then split the population into test portions and control portions.

In particular embodiments, adding inositol that has been labeled with a radioisotope to the test cells and the control cells is done by growing or incubating the test cells and control cells in inositol-free medium and then adding radiolabeled inositol to the medium or changing the medium to a medium that contains radiolabeled inositol. Preferably, the cells are grown or incubated in the presence of the radiolabeled inositol for about 4 to 40 hr, even more preferably for about 8 to 36 hr, and most preferably for about 16 to 24 hr.

In particular embodiments, the test cells and control cells are present in the wells of a multiwell microtiter plate.

In particular embodiments, the inositol is radiolabeled with ³H or ¹⁴C. In particular embodiments, the inositol is radiolabeled with ³H, the cells are present in microtiter plates, and the amount of ³H added to each well of the microtiter plates is from about 0.1 μCi to about 10 μCi, preferably from about 0.5 μCi to about 5 μCi, and most preferably about 1 μCi.

In particular embodiments, the incubations of steps (b) and (g) are carried out for a period of from 30 seconds to 24 hr, preferably from 30 min to 10 hr, even more preferably from 1 hr to 4 hrs, and most preferably for about 1 hr.

In particular embodiments, the test and control cells are lysed by a process that involves cycling the cells between a relatively low (e.g., −80° C.) and a relatively high (e.g., 37° C.) temperature, i.e., freeze/thawing. Alternatively, the test and control cells may be lysed by treatment with detergent. In particular embodiments, the lysing occurs in the presence of formic acid, preferably at a concentration of from 0.05 M to 0.1 M. Most simply, the cells can be lysed by merely adding formic acid and agitating (i.e., without freeze/thawing or detergent). The final concentration of formic acid should be from about 20 mM to about 200 mM. For example, if the cells are present in the wells of a 96-well microtiter plate, one could add 200 μl of a stock solution of 0.2 M formic acid to each well. Agitation can be accomplished by the use of a plate shaker and is generally carried out for about 5 minutes at room temperature, although agitating for longer periods is also suitable.

In particular embodiments, a glass bead doped with Ce, Mn, Cu, Pb, Sn, Au, Ag, or Sm is used as the solid phase. In particular embodiments, the solid phase is yttrium silicate doped with Ce (Y₂SiO₅:Ce) formed into beads. In particular embodiments, the test and control lysates are brought in contact with the solid phase by mixing a portion of the lysates with a suspension of beads formed from a glass doped with Ce, Mn, Cu, Pb, Sn, Au, Ag, or Sm, preferably Y₂SiO₅:Ce. In particular embodiments, the mixture of lysates and beads is incubated for a time sufficient to allow the beads to settle out by gravity. Allowing for such settling of the beads can in some circumstances reduce the variance of the data obtained.

In particular embodiments, the amount of radioactivity adhered to the solid phase is determined by adding the solid phase to scintillation fluid and counting the fluid and solid phase in a scintillation counter. Alternative ways of measuring scintillation include the use of those imaging systems that are sensitive enough to record the low level of light emission from scintillation proximity assays. An example of such a system is the LEADSEEKER® (Amersham Pharmacia Biotech, Amersham, UK), see Ramm, 1999, Drug Discovery Today 4:401-410.

A wide variety of cell lines can be used in the present invention. Particularly preferred are mammalian cell lines. In particular embodiments, the test cells and control cells are selected from the group consisting of: L cells L-M(TK⁻) (ATCC CCL 1.3), L cells L-M (ATCC CCL 1.2), HEK293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), T24 (ATCC HTB4), and MRC-5 (ATCC CCL 171).

The present invention also provides methods of identifying substances that are agonists or antagonists of G-protein coupled receptors (GPCRs) where the GPCRs are coupled to the inositol phosphate pathway.

In general terms, the methods of identifying agonists can be practiced as follows. Test cells expressing a GPCR coupled to the inositol phosphate pathway are grown or incubated in medium containing no inositol. The medium is then supplemented with inositol that has been labeled with a radioisotope and the test cells are cultured for a period sufficient to permit the uptake of the labeled inositol into the test cells such that a portion of the inositol and inositol phosphates in the test cells becomes labeled. The medium is replaced with fresh medium without inositol, followed by or together with the addition of a substance that is to be tested as a possible agonist of the GPCR. The test cells are incubated with the substance for a period sufficient for the substance to activate the GPCR in the test cells if the substance is in fact an agonist of the GPCR. This leads to an increase in the intracellular concentration of inositol phosphates relative to inositol. Usually, incubation of the test cells with the substance is carried out in the presence of lithium chloride (LiCl). LiCl is an inhibitor of inositol phosphatases and its presence prevents conversion of inositol phosphate to inositol, thus making the readout a quantitative measure of GPCR activation. The medium is then removed, the test cells are lysed, and test lysates are prepared. Control lysates are also prepared from control cells that are essentially the same as the test cells and that have been treated in the same manner as the test cells, except that the control cells are not exposed to the substance. The test and control lysates, containing radiolabeled inositol phosphates and radiolabeled inositol, are brought into contact with an appropriate solid phase such as a scintillation bead with a positive surface charge. The lysates and the solid phase are incubated to allow inositol phosphates in the lysates to bind to the solid phase while inositol remains in the lysate. The resultant scintillation from the solid phase, due to the adhered inositol phosphates, is detected by a suitable instrument. If the substance is an agonist of the GPCR, the substance will have activated the GPCR and caused an increase in the concentration of labeled inositol phosphates in the test cells. This increase will not have occurred in the control cells since the control cells will not have been exposed to the substance. Thus, the level of labeled inositol phosphates in the test cells will have been greater than the level of labeled inositol phosphates in the control cells. This will be reflected in the lysates, with the lysate from the test cells having a higher level of labeled inositol phosphates than the lysate from the control cells. Therefore, there will be more radioactivity (i.e., scintillation) detected from the test lysate than from the control lysate if the substance is an agonist.

Accordingly, the present invention provides a method of identifying agonists of a G-protein coupled receptor (GPCR) comprising:

(a) adding inositol that has been labeled with a radioisotope to test cells expressing the GPCR so that the inositol that has been labeled with a radioisotope is incorporated into inositol and inositol phosphates in the test cells;

(b) incubating the test cells with a substance for a period sufficient for the substance to activate the GPCR in the test cells;

(c) lysing the test cells and preparing a test lysate from the test cells;

(d) adding the test lysate to a solid phase so that inositol phosphates from the test lysate adhere to the solid phase while inositol from the test lysate does not adhere to the solid phase;

(e) determining the amount of radioactivity adhered to the solid phase in step (d);

(f) adding inositol that has been labeled with a radioisotope to control cells expressing the GPCR so that the inositol that has been labeled with a radioisotope is incorporated into inositol and inositol phosphates in the control cells;

(g) incubating the control cells in the absence of the substance for a period essentially the same as the period in step (b);

(h) lysing the control cells and preparing a control lysate from the control cells;

(i) adding the control lysate to a solid phase so that inositol phosphates from the control lysate adhere to the solid phase while inositol from the control lysate does not adhere to the solid phase;

(j) determining the amount of radioactivity adhered to the solid phase in step (i);

where if the amount of radioactivity determined in step (e) is greater than the amount of radioactivity determined in step (j) then the substance is an agonist of the G-protein coupled receptor.

The step of determining the amount of radioactivity adhered to the solid phase of steps (e) and (j) can be conveniently carried out by measuring the total amount of radioactivity (e.g., by scintillation counting) in the mixtures of lysates and solid phases since essentially all of the scintillation results from the radioactivity adhered to the beads (i.e., from the inositol phosphates) and very little scintillation results from the radioactivity of the inositol in the solution phase of the lysates.

For the sake of convenience, the steps of adding labeled inositol to the test cells and to the control cells (steps (a) and (f), respectively) can be carried out at the same time. That is, one can label a single population of cells with inositol, then split the population into test portions and control portions.

In particular embodiments, adding inositol that has been labeled with a radioisotope to the test cells and the control cells is done by growing or incubating the test cells and control cells in inositol-free medium and then adding radiolabeled inositol to the medium or changing the medium to a medium that contains radiolabeled inositol. Preferably, the cells are grown or incubated in the presence of the radiolabeled inositol for about 4 to 40 hr, even more preferably for about 8 to 36 hr, and most preferably for about 16 to 24 hr.

In particular embodiments, the test cells and control cells are present in the wells of a multiwell microtiter plate.

In particular embodiments, the inositol is radiolabeled with ³H or ¹⁴C. In particular embodiments, the inositol is radiolabeled with ³H, the cells are present in the wells of a microtiter plate, and the amount of ³H added to each well of the microtiter plates is from about 0.1 μCi to about 10 μCi, preferably from about 0.5 μCi to about 5 μCi, and most preferably about 1 μCi.

In particular embodiments, the incubations of steps (b) and (g) are carried out for a period of from 30 seconds to 24 hr, preferably from 10 min to 10 hr, even more preferably from 1 hr to 4 hrs, and most preferably for about 1 hr.

In particular embodiments, the test and control cells are lysed by a process that involves cycling the cells between a relatively low (e.g., −80° C.) and a relatively high (e.g., 37° C.) temperature, i.e., freeze/thawing. Alternatively, the test and control cells may be lysed by treatment with detergent. In particular embodiments, the lysing occurs in the presence of formic acid, preferably at a concentration of from 0.05 M to 0.1 M. Most simply, the cells can be lysed by merely adding formic acid and agitating (i.e., without freeze/thawing or detergent). The final concentration of formic acid should be from about 20 mM to about 200 mM. For example, if the cells are present in the wells of a 96-well microtiter plate, one could add 200 μl of a stock solution of 0.2 M formic acid to each well. Agitation can be accomplished by the use of a plate shaker and is generally carried out for about 5 minutes at room temperature, although agitating for longer periods is also suitable.

In particular embodiments, a glass bead doped with Ce, Mn, Cu, Pb, Sn, Au, Ag, or Sm is used as the solid phase. In particular embodiments, the solid phase is yttrium silicate doped with Ce (Y₂SiO₅:Ce) formed into beads. In particular embodiments, the test and control lysates are brought in contact with the solid phase by mixing a portion of the lysates with a suspension of beads formed from a glass doped with Ce, Mn, Cu, Pb, Sn, Au, Ag, or Sm, preferably Y₂SiO₅:Ce. In particular embodiments, the mixture of lysates and beads is incubated for a time sufficient to allow the beads to settle out by gravity. Allowing for such settling of the beads can in some circumstances reduce the variance of the data obtained.

In particular embodiments, the amount of radioactivity adhered to the solid phase is determined by adding the solid phase to scintillation fluid and counting the fluid and solid phase in a scintillation counter.

In particular embodiments, LiCl to a final concentration of about 0.5 mM to 20 mM, preferably about 1 mM to 15 mM, and even more preferably 5 mM to 10 mM is added at steps (b) and (g).

In particular embodiments, the test cells and control cells naturally express the GPCR. In other embodiments, the test cells and control cells do not naturally express the GPCR but have been transfected, either transiently or stably, with an expression vector encoding the GPCR so that the GPCR is expressed in the test cells and control cells. In certain embodiments, the test cells and control cells have been transfected so as to express a chimeric or promiscuous Gα subunit, thereby coupling the GPCR to the inositol phosphate pathway.

In particular embodiments, the test cells and control cells are selected from the group consisting of: L cells L-M(TK⁻) (ATCC CCL 1.3), L cells L-M (ATCC CCL 1.2), HEK293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), N]H3T3 (ATCC CRL 1658), HeLa (ATCC CCL 2), C1271 (ATCC CRL 1616), BS-C-1 (ATCC CCL 26), T24 (ATCC HTB-4), and MRC-5 (ATCC CCL 171).

When using the methods of the present invention to screen for agonists of GPCRs, it will often be desirable to ensure that the substances identified are specific for the GPCRs of interest. This can be accomplished by running additional controls to those specified above. Such additional controls would entail carrying out the steps of the method but using cells that are substantially identical to the test cells as control cells except that the additional control cells do not express the GPCR of interest. The additional control cells would be exposed to the substance in the same manner as the test cells. One possibility would be to use non-recombinant parent cells as the additional control cells where the test cells express the GPCR of interest due to the recombinant expression of the GPCR.

The above-described additional controls can be used to confirm the identity of substances that score as hits in the methods described above. Alternatively, methods based on such additional controls can be used as primary screens.

Accordingly, the methods of the present invention include a method of identifying agonists of a G-protein coupled receptor (GPCR) comprising:

(a) adding inositol that has been labeled with a radioisotope to test cells expressing the GPCR so that the inositol that has been labeled with a radioisotope is incorporated into inositol phosphates in the test cells;

(b) incubating the test cells with a substance for a period sufficient for the substance to activate the GPCR in the test cells;

(c) lysing the test cells and preparing a test lysate from the test cells;

(d) adding the test lysate to a solid phase so that inositol phosphates from the test lysate adhere to the solid phase while inositol from the test lysate does not adhere to the solid phase;

(e) determining the amount of radioactivity adhered to solid phase in step (d);

(f) adding inositol that has been labeled with a radioisotope to control cells that are substantially identical to the test cells except that the control cells do not express the GPCR so that the inositol that has been labeled with a radioisotope is incorporated into inositol and inositol phosphates in the control cells;

(g) incubating the control cells with the substance for a period essentially the same as the period in step (b);

(h) lysing the control cells and preparing a control lysate from the control cells;

(i) adding the control lysate to a solid phase so that inositol phosphates from the control lysate adhere to the solid phase while inositol from the control lysate does not adhere to the solid phase;

(j) determining the amount of radioactivity adhered to the solid phase in step (i);

where if the amount of radioactivity determined in step (e) is greater than the amount of radioactivity determined in step (j) then the substance is an agonist of the G-protein coupled receptor.

The methods described herein for identifying agonists of GPCRs can be modified so as to identify antagonists of GPCRs. The test cells are exposed to a known agonist of the GPCR in addition to the substance. The known agonist will cause an increase in the level of inositol phosphates measured from the test cells if the substance has no effect on the GPCR. If the substance is an antagonist of the GPCR, it will be capable of preventing or diminishing this increase in inositol phosphates caused by the known agonist.

Accordingly, the present invention provides a method of identifying antagonists of a G-protein coupled receptor (GPCR) comprising:

(a) adding inositol that has been labeled with a radioisotope to test cells expressing the GPCR so that the inositol that has been labeled with a radioisotope is incorporated into inositol and inositol phosphates in the test cells;

(b) incubating the test cells with a known agonist of the GPCR and a substance for a period sufficient for the agonist to activate the GPCR in the test cells if the substance is not an antagonist;

(c) lysing the test cells and preparing a test lysate from the test cells;

(d) adding the test lysate to a solid phase so that inositol phosphates from the test lysate adhere to the solid phase while inositol from the test lysate does not adhere to the solid phase;

(e) determining the amount of radioactivity adhered to the solid phase in step (d);

(f) adding inositol that has been labeled with a radioisotope to control cells expressing the GPCR so that the inositol that has been labeled with a radioisotope is incorporated into inositol and inositol phosphates in the control cells;

(g) incubating the control cells in the presence of the agonist but in the absence of the substance for a period essentially the same as the period in step (b);

(h) lysing the control cells and preparing a control lysate from the control cells;

(i) adding the control lysate to a solid phase so that inositol phosphates from the control lysate adhere to the solid phase while inositol from the control lysate does not adhere to the solid phase;

(j) determining the amount of radioactivity adhered to the solid phase in step (i);

where if the amount of radioactivity determined in step (i) is greater than the amount of radioactivity determined in step (e) then the substance is an agonist of the GPCR.

One skilled in the art would recognize that, where the present invention involves comparing control values for the level of inositol phosphates to test values for the level of inositol phosphates and determining whether the control values are greater or less than the test values, a non-trivial difference is sought. For example, if in the method of identifying antagonists of GPCRs described immediately above, the control value were found to be 1% greater than the test value, this would not indicate that the substance is an antagonist. Rather, one skilled in the art would attribute such a small difference to normal experimental variance. What is looked for is a significant difference between control and test values. For the purposes of this invention, a significant difference fulfills the usual requirements for a statistically valid measurement of a biological signal. For example, depending upon the details of the experimental arrangement, a significant difference might be a difference of at least 10%, prefereably at least 20%, more preferably at least 50%, and most preferably at least 100%.

Before development of the methods described herein, measurement of cellular inositol phosphates was usually accomplished by labeling cells with tritiated inositol, followed by preparation of a cell extract. Radiolabeled inositol phosphates were then resolved from radiolabeled inositol by anion exchange chromatography followed by measurement by scintillation counting. This method could not be used in automated high-throughput screening because of the column chromatography step. Thus, the column chromatography step was a significant disadvantage to the prior methods. One advantage of the present methods is that the use of the solid phase to discriminate between radiolabeled inositol and radiolabeled inositol phosphate removes the requirement for a chromatography step. Therefore, the methods can be readily automated and miniaturized, making them suitable for high-throughput screening.

The present invention employs cells expressing inositol phosphate phosphatases for which it is desired to identify inhibitors or cells expressing GPCRs for which it is desired to identify agonists or antagonists. Such cells are generally produced by transfecting cells that do not normally express the inositol phosphate phosphatases or GPCRs with expression vectors encoding the inositol phosphate phosphatases or GPCRs and then culturing the cells under conditions such that functional inositol phosphate phosphatases or GPCRs are formed. In this way, recombinant host cells expressing functional inositol phosphate phosphatases or GPCRs are produced. In some embodiments, the present invention may also employ cell lines that naturally express the inositol phosphate phosphatases or GPCRs.

Recombinant host cells for use in the present invention are preferably eukaryotic cells, including but not limited to, cell lines of human, bovine, porcine, monkey and rodent origin. Cells and cell lines which are suitable for recombinant expression, many of which are commercially available, include but are not limited to, L cells L-M(TK⁻) (ATCC CCL 1.3), L cells L-M (ATCC CCL 1.2), HEK293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), T24 (ATCC HTB-4), and MRC-5 (ATCC CCL 171).

In certain embodiments of the present invention, where the cells used do not naturally express the inositol phosphate phosphatase or GPCR of interest, and DNA encoding the inositol phosphate phosphatase or GPCR is transfected into the cells, in order to express the inositol phosphate phosphatase or GPCR in the cells, DNA encoding the inositol phosphate phosphatase or GPCR can be obtained by methods well known in the art. For example, a cDNA fragment encoding the inositol phosphate phosphatase or GPCR can be isolated from a suitable cDNA library by using the polymerase chain reaction (PCR) employing suitable primer pairs. The cDNA fragment encoding the inositol phosphate phosphatase or GPCR can then be cloned into a suitable expression vector. Primer pairs can be selected based upon the known DNA sequence of the inositol phosphate phosphatase or GPCR it is desired to obtain. Suitable cDNA libraries can be made from cellular or tissue sources known to contain mRNA encoding the inositol phosphate phosphatase or GPCR.

One skilled in the art would know that for certain GPCRs in certain cell types, it is desirable to co-transfect, and thereby express, particular G-protein subunits in order to obtain a functional ion channel. Common knowledge in the art will lead the skilled artisan to express the correct G-protein subunits in the transfected cells.

GPCRs transmit signals across cell membranes upon the binding of ligand. The ligand-bound GPCR interacts with a heterotrimeric G-protein, causing the Gα subunit of the G-protein to disassociate from the Gβ and Gγ subunits. The Gα subunit can then go on to activate a variety of second messenger systems. Generally, a particular GPCR is only coupled to a particular type of G-protein a subunit (e.g., GαI, Gαq, or Gαo). GPCRs that couple to Gαi generally are much less efficient at activating phospholipase C (and thus the inositiol phosphate synthetic pathway) than GPCRs that couple to other subunits (e.g., Gαq or Gαo). However, it has been found that Gi-coupled receptors can be studied via activation of phospholipase C and its consequent production of inositol phosphate if those Gi-coupled GPCRs are co-expressed with certain chimeric or promiscuous G-protein subunits. The chimeric G-protein αaqi5 binds to Gi-coupled receptors via its carboxyl end and activates phospholipase C via its Gαq portion. The promiscuous G-proteins Gα15 and Gα16 can be used to couple virtually any GPCR to the inositol phosphate pathway. See, e.g., Conklin et al., 1993, Nature 363:274-276; Coward et al., 1999, Anal. Biochem. 270:242-248; Gomeza et al., 1996, Mol. Pharmacol. 50:923-930; Offermanns & Simon, 1995, J. Biol. Chem. 270:15175-15180. Thus, when Gi-coupled receptors are co-expressed in cells with Gαqi5, Gα15, or Gα16, the GPCR's activation can be monitored via an inositol phosphate assay such as those described herein.

One skilled in the art could use published inositol phosphate phosphatase or GPCR sequences to design PCR primers and published studies of inositol phosphate phosphatase or GPCR expression to select the appropriate sources from which to make cDNA libraries in order to obtain DNA encoding the inositol phosphate phosphatase or GPCR. The following publications may be of use in this regard:

McAllister et al., 1992, Biochem. J. 284:749-754 describe the cDNA cloning of human and rat brain myo-inositol monophosphatase as well as the expression and characterization of the human recombinant enzyme. See GenBank accession no. X66922.

York et al., 1993, J. Biol. Chem. 90:5833-5837 describe the cloning, heterologous expression, and chromosomal localization of human inositol polyphosphate 1-phosphatase. See GenBank accession no. L08488.

Attree et al., 1992, Nature 358:239-242 discloses the Lowe's oculocerebrorenal syndrome gene, which encodes a protein highly homologous to inositol polyphosphate-5-phosphatase. See GenBank accession no. M88162.

Norris et al., 1997, J. Biol. Chem. 272:23859-23864 describes the cDNA cloning and characterization of inositol polyphosphate 4-phosphatase type II. See GenBank accession no. NM003866.

Takahashi et al., 1992, Eur. J. Biochem. 204:1025-1033 discloses the primary structure and gene organization of human substance P and neuromedin K receptors. See GenBank accession X65181.

Desai et al., 1995, Mol. Pharmacol. 48:648-657 describes the cloning and expression of a human metabotropic glutamate receptor 1 alpha. See GenBank accession no. NM000838.

Vu et al., 1991, Cell 64:1057-1068 describes the cloning of a functional thrombin receptor. See GenBank accession no. M62424.

Bonner et al., 1988, Neuron 1:403-410 describes the cloning and expression of the human and rat m5 muscarinic acetylcholine receptor genes. See Genbank accession no. U29589.

Morse et al., 2001, J. Pharmacol. Exp. Ther. 29:1058-1066 describes the cloning and characterization of a novel human histamine receptor. See Genbank accessionno. AF329449.

PCR reactions can be carried out with a variety of thermostable enzymes including but not limited to AmpliTaq, AmpliTaq Gold, or Vent polymerase. For AmpliTaq, reactions can be carried out in 10 mM Tris-Cl, pH 8.3, 2.0 mM MgCl₂, 200 μM of each DNTP, 50 mM KCl, 0.2 μM of each primer, 10 ng of DNA template, 0.05 units/μl of AmpliTaq. The reactions are heated at 95° C. for 3 minutes and then cycled 35 times using suitable cycling parameters, including, but not limited to, 95° C., 20 seconds, 62° C., 20 seconds, 72° C., 3 minutes. In addition to these conditions, a variety of suitable PCR protocols can be found in PCR Primer, A Laboratory Manual, edited by C. W. Dieffenbach and G. S. Dveksler, 1995, Cold Spring Harbor Laboratory Press; or PCR Protocols: A Guide to Methods and Applications, Michael et al., eds., 1990, Academic Press.

It is desirable to sequence the DNA encoding the inositol phosphate phosphatase or GPCR obtained by the herein-described methods, in order to verify that the desired the inositol phosphate phosphatase or GPCR has in fact been obtained and that no unexpected changes have been introduced into its sequence by the PCR reactions. The DNA can be cloned into suitable cloning vectors or expression vectors, e.g., the mammalian expression vector pcDNA3.1 (Invitrogen, San Diego, Calif.) or other expression vectors known in the art or described herein.

A variety of expression vectors can be used to recombinantly express DNA encoding inositol phosphate phosphatases or GPCRs for use in the present invention. Commercially available expression vectors which are suitable include, but are not limited to, pMClneo (Stratagene), pSG5 (Stratagene), pcDNAI and pcDNAIamp, pcDNA3, pcDNA3.1, pCR3.1 (Invitrogen, San Diego, Calif.), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pCI.neo (Promega), pTRE (Clontech, Palo Alto, Calif.), pV1Jneo, pIRESneo (Clontech, Palo Alto, Calif.), pCEP4 (Invitrogen, San Diego, Calif.), pSC11, and pSV2-dhfr (ATCC 37146). The choice of vector will depend upon cell type in which it is desired to express the inositol phosphate phosphatase or GPCR, as well as on the level of expression desired, and the like.

The expression vectors can be used to transiently express or stably express the inositol phosphate phosphatase or GPCR. The transient expression or stable expression of transfected DNA is well known in the art. See, e.g., Ausubel et al., 1995, “Introduction of DNA into mammalian cells,” in Current Protocols in Molecular Biology, sections 9.5.1-9.5.6 (John Wiley & Sons, Inc.).

As an alternative to the above-described PCR methods, cDNA clones encoding inositol phosphate phosphatases or GPCRs can be isolated from cDNA libraries using as a probe oligonucleotides specific for the desired inositol phosphate phosphatase or GPCR and methods well known in the art for screening cDNA libraries with oligonucleotide probes. Such methods are described in, e.g., Sambrook et al., 1989, Molecular Cloning: A Laboratory Manual; Cold Spring Harbor Laboratory, Cold Spring Harbor, N.Y.; Glover, D. M. (ed.), 1985, DNA Cloning: A Practical Approach, MRL Press, Ltd., Oxford, U.K., Vol. I, II. Oligonucleotides that are specific for particular inositol phosphate phosphatases or GPCRs and that can be used to screen cDNA libraries can be readily designed based upon the known DNA sequences of the inositol phosphate phosphatases or GPCRs and can be synthesized by methods well-known in the art.

If desired, methods of alleviating color quenching, which can attenuate the signals of the assays described herein, can be employed. Such methods are known in the art. For example, methods of color quenching are described in International Patent Publication WO 99/09415.

The present invention extends the advantages of scintillation proximity assays to measurements of inositol phosphate levels. The simplicity of the invention allows for the almost complete automation of the assay using robotic sample processors and microtiter plate scintillation counters. As a result, the assays of the present invention are capable of high throughput, and therefore are highly useful for screening drug candidates.

The following non-limiting examples are presented to better illustrate the invention.

### EXAMPLE 1

YSi SPA Beads Preferentially Detect Inositol Phosphate Over Inositol

10 μl of either 100 μM ³H-inositol or 100 μM ³H-inositol-1-phosphate in 1 mM ammonium phosphate, pH 8 (specific activity 1 nCi/μl) was mixed with the yttrium silicate scintillation proximity assay (YSi SPA) beads sold by Amersham Pharmacia Biotech (catalog no. RPNQ0013). As a control, the same amount of ³H-inositol or ³H-inositol-1-phosphate was directly mixed with Microscint-20 (Packard) without first being exposed to the YSi SPA beads.

The YSi SPA beads were supplied by Amersham as a slurry at 100 mg/ml in water. The tests were carried out in the wells of a 96-well microfiter plate (Picoplate-96, Packard). 1 mg of the YSi SPA bead slurry was used per well. The test mixtures contained, added in this order to the wells: 10 μl SPA beads, 60 μl water, 20 μl 100 mM formic acid, and 10 μl of 100 μM of either ³H-inositol or ³H-inositol-1-phosphate in 1 mM ammonium phosphate, pH 8.0. Each well was performed in duplicate. In another plate, the same amount of radiolabeled inositol or inositol-1-phosphate was added to wells followed by 100 μl Microscint-20 (Packard). Plates were sealed using Topseal-A (Packard), and agitated for 1 hr at speed 7 on a commercial titer plate shaker (Labline Instruments Inc.) at room temperature. Plates were then allowed to sit at room temperature for 2 hr before counting.

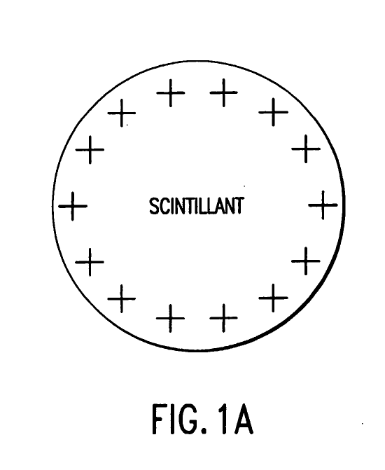

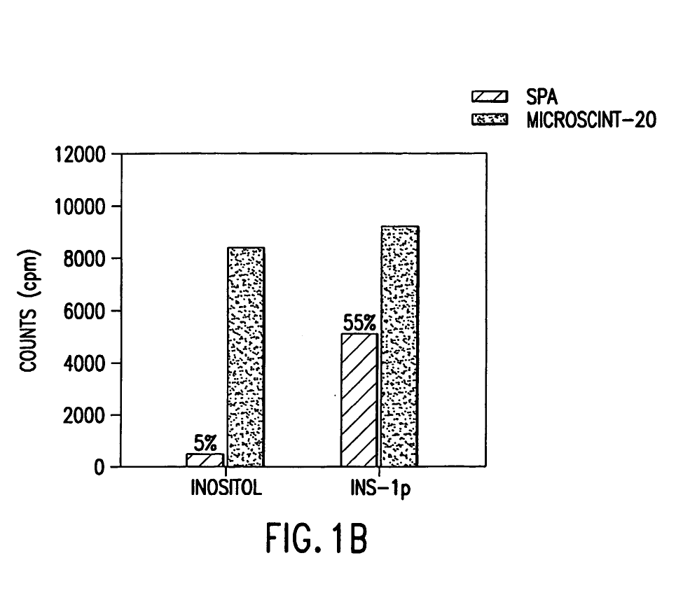

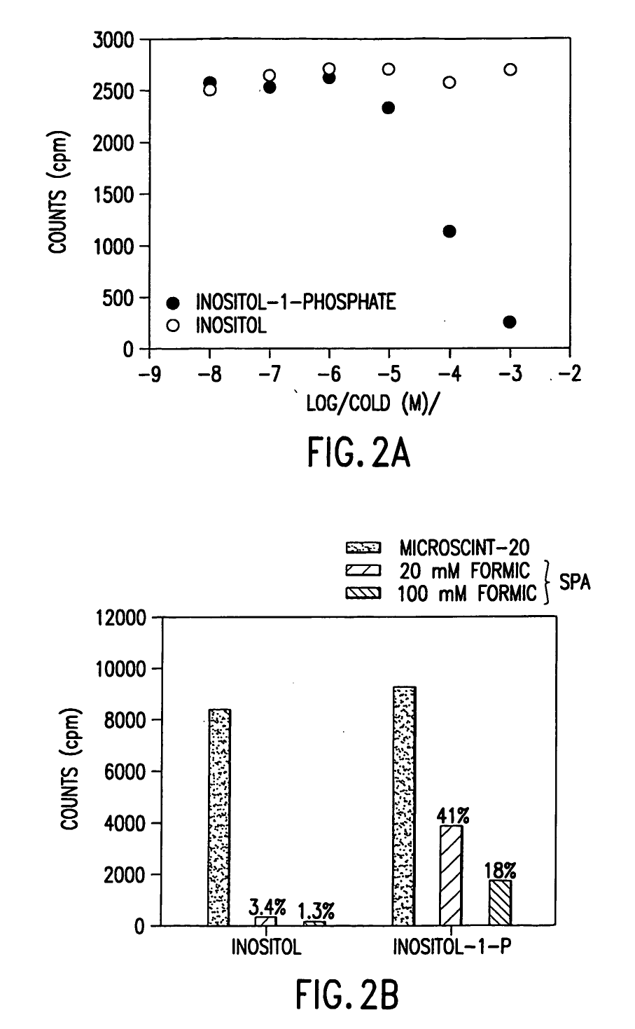

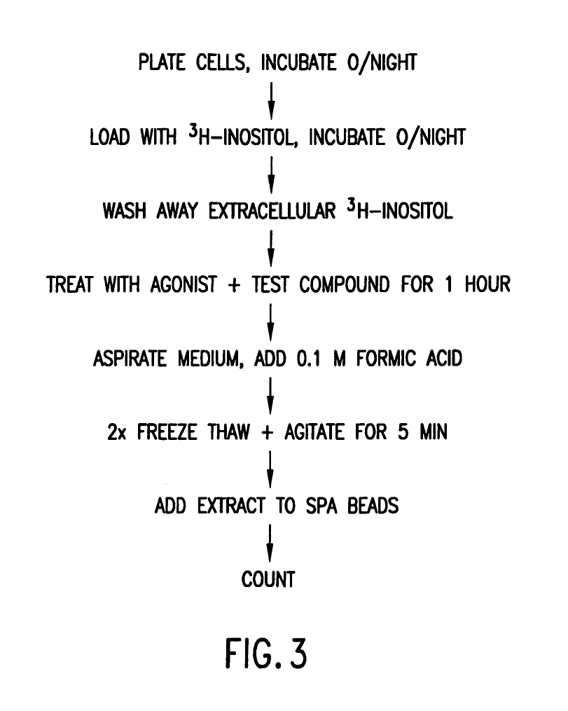

The results are shown in FIG. 1B. The efficiency of detection of ³H-inositol-1-phosphate by YSi SPA beads under the conditions described was 55% relative to detection using Microscint-20, compared to an efficiency of only 5% for detection of ³H-inositol.

In the above, ammonium phosphate was included in the mixture because the ³H-inositol-1-phosphate was supplied by the manufacturer (New England Nuclear) in aqueous solution containing 10 mM ammonium phosphate, pH 8.0. Therefore, since the ammonium phosphate was carried through to the mixture with the SPA beads, the same concentration was added to the ³H-inositol test to keep the conditions the same.

### EXAMPLE 2

Preparation of M1-CHO Cells

M1-CHO cells are prepared according to the methods described in Example 9 for M1-T24 cells. Also, CHO cells expressing the M1 muscarinic acetylcholine receptor are widely available and can be used in the methods of the present invention.

### EXAMPLE 3

Assay for Activation of the M1 Muscarinic Receptor

M1-CHO cells were plated in Falcon 353072 96-well tissue culture plates in Ham's F12 glutamax supplemented with 10% fetal bovine serum (FBS) and 100 μg/ml streptomycin, 100 units/ml penicillin (Gibco-BRL, Gaithersburg, Md.). 4×10⁵ cells in 100 μl per well were plated as in Example 12 using the repeat mode of a Biohit pipettor on the slowest speed. The cells were grown at 37° C. until they were about 90% confluent.

The media was aspirated from the wells and 200 μl per well of DMEM without inositol (Gibco-BRL 11968-021) prewarmed to 37° C. was added. The cells were then washed an additional time with 200 μl per well of the DMEM without inositol. Care was taken during the aspiration steps so that as few cells as possible are dislodged. To this end, the same portion of the bottom of the well was touched at each aspiration.

Following the last aspiration, to each well was added 100 μl of the DMEM without inositol. To each well was then added 100 μl of DMEM without inositol supplemented with 0.6% bovine serum albumin (BSA), and ³H-inositol to a specific activity of 10 μCi/ml. The cells were incubated with the label overnight (about 22 hr) at 37° C. After the overnight incubation, the cells appeared healthy, about 80-90% confluent, with few floaters or rounded up cells.

A dilution plate was prepared containing 3× solutions of DMEM without inositol but with 0.3% BSA and various additions. The solutions contained either (a) carbachol; (b) carbachol plus lithium chloride; (c) lithium chloride; or (d) no additions.

The wells containing the M1-CHO cells were washed twice with 200 μl of DMEM without inositol but supplemented with 0.3% bovine serum albumin (BSA) and prewarmed to 37° C. Then 100 μg of this DMEM was added per well. This was followed with 50 μl of the appropriate solution from the dilution plate and the cells were incubated for 1 hr at 37° C. The final concentration of carbachol when included was 1 mM. The final concentration of LiCl when included was 5 mM.

The medium was aspirated from the wells, 200 μl of 0.1 M formic acid was added to each well, the plate was sealed, and then stored at −80° C. The cells were lysed by being placed on a heating block and subjected to two cycles of 20 minutes at −80° C. and then 20 minutes at 37° C. Then the plates were then shaken at speed 7 for 5 minutes on a filter plate shaker.

Although the cells were clearly lysed at this point, some particulate matter was present in the bottoms of the wells. 200 μl was removed from each well, with care taken not to include any of the particulate matter. The 200 μl aliquots were transferred to the wells of a V-bottom plate and triturated 3×to mix.

As controls, 10 μl of each aliquot was mixed with Microscint-20 and counted in a scintillation counter.

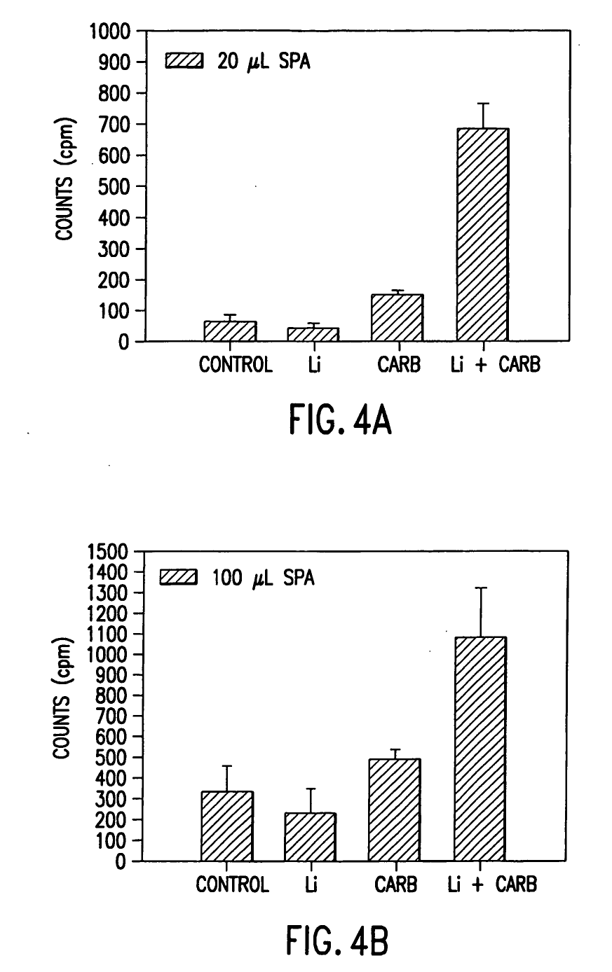

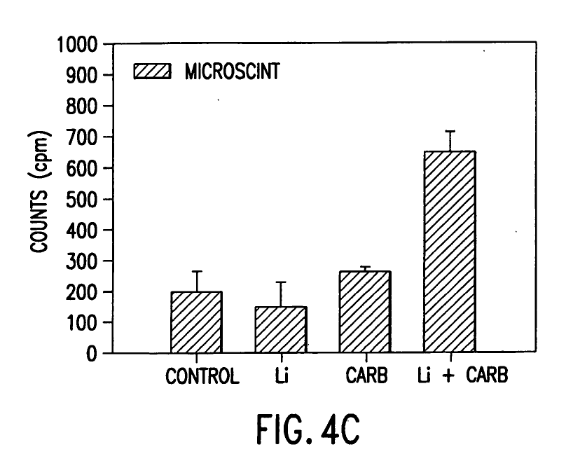

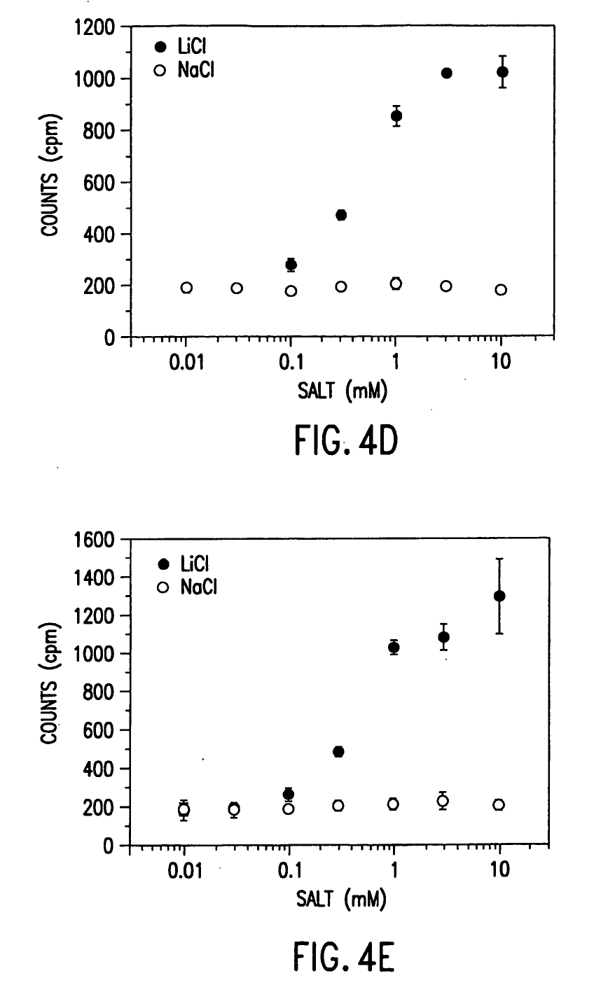

20 μl of each aliquot was mixed with 80 μl of H₂O and 10 μl of YSi SPA beads. The mixture was shaken for 1 hr at speed 7 on a titer plate shaker and then allowed to settle for 2 hr prior to counting. The results are shown in FIG. 4A.

100 μl of each aliquot was mixed 10 μl of YSi SPA beads. The mixture was shaken for 1 hr at speed 7 on a titer plate shaker and then allowed to settle for 2 hr prior to counting. The results are shown in FIG. 4B.

### EXAMPLE 4

Assay for the Identification of Agonists and Antagonists of the Luteinizing Hormone Releasing Hormone (LHRH) Receptor

The coding sequence of the human LHRH receptor is isolated by PCR, inserted into a suitable expression vector (e.g., pcDNA (Invitrogen, Carlsbad, Calif.)), and transfected into HEK-293 cells (ATCC CRL 1573) to form a cell line expressing LHRH receptor as described in Lin et al., 1995, Mol. Pharmacol. 47:131-139. HEK-293 cells are cultured in DMEM with 10% fetal bovine serum, 4 mM glutamine and suitable antibiotics/antimycotics. Selection of transfected cells is done with G418 and the transfected cells are maintained in 550 μg/ml of G418. To confirm that transfected cells surviving in G418 actually express the LHRH receptor, immunoprecipitation assays are performed using suitable antisera or monoclonal antibodies that are specific for the LHRH receptor.

HEK-293 cells expressing LHRH receptor are plated into 96-well microtiter plates at about 2.5×10⁴ cells per well and cultured overnight. The cells are then washed in inositol-free DMEM and incubated about 20 hr in inositol-free DMEM with 0.3% bovine serum albumin supplemented with 0.80 μCi/well of myo-1,2-³H-inositol. The cells are then washed once in inositol-free DMEM with 0.3% bovine serum albumin supplemented with 5 mM lithium chloride and various concentrations of potential agonists are added to the wells for about 1 hr. At the end of this period, the medium is removed and the cells are lysed and analyzed as in Example 3.

In a variation of the above method, antagonists of the LHRH receptor are identified by adding, instead of the potential agonists, a known agonist (e.g., LHRH, [D-trp⁶]LHRH, at about 10⁻⁹ to 10⁻¹⁰ M) together with potential antagonists. Positive control wells treated with known agonists alone (no potential antagonists) are run. If the potential antagonists really are antagonists, their presence should decrease the amount of inositol phosphates produced by stimulation of the LHRH receptor with the known agonist alone.

### EXAMPLE 5

Assay for the Identification of Agonists and Antagonists of the Human Neurokinin 1 (NK1) Receptor

The human NK1 receptor is cloned and expressed in CHO cells (ATCC CCL 61) as described in Chung et al., 1994, Biochem. Biophys. Res. Comm. 198:967-972. Cells expressing the NK1 receptor are plated into 96-well microtiter plates at about 1×10⁴ cells per well and cultured overnight. The medium is changed to EMEM/F12 (with Earle's salt) containing 10 μCi/ml of [³H]-myo-inositol and the cells are incubated for about 16-24 hr to allow for the uptake of the [³H]-myo-inositol and incorporation into phosphatidyl inositol. The inositol containing medium is removed and the cells are washed twice with assay buffer (MEM containing 10 mM LiCl, 20 mM HEPES, and 1 mg/ml BSA). The cells are then incubated for 20 min at 37° C. in assay buffer. Various concentrations of potential agonists are added to the wells for about 1 hr. At the end of this period, the medium is removed and the cells are lysed and analyzed as in Example 3.

### EXAMPLE 6

Assay for the Identification of Agonists and Antagonists of the Human Neurokinin 3 (NK3) Receptor

The human NK3 receptor is cloned and expressed in CHO cells (ATCC CCL 61) as described in Tian et al., 1996, J. Neurochem. 67:1191-1199. Cells expressing the NK3 receptor are plated into 96-well microtiter plates at about 1×10⁴ cells per well and cultured overnight. The medium is changed to EMEM/F12 (with Earle's salt) containing 10 μCi/ml of [³H]-myo-inositol and the cells are incubated for about 16-24 hr to allow for the uptake of the [³H]-myo-inositol and incorporation into phosphatidyl inositol. The inositol containing medium is removed and the cells are washed twice with assay buffer (MEM containing 10 mM LiCl, 20 mM HEPES, and 1 mg/ml BSA). The cells are then incubated for 20 min at 37° C. in assay buffer. Various concentrations of potential agonists are added to the wells for about 1 hr. At the end of this period, the medium is removed and the cells are lysed and analyzed as in Example 3.

### EXAMPLE 7

Assay for the Identification of Agonists and Antagonists of the Human Chemokine Receptor CCR2b

The human CCR2b receptor is cloned and expressed in COS-7 cells (ATCC CCL 1651) along with the G-protein subunit Gα₁₄ as described in Le Gouill et al., 1999, J. Biol. Chem. 274:12548-12554. Cells expressing the human CCR2b receptor are plated in Dulbecco's Modified Eagle's Medium (DMEM) high glucose (Life Technologies, Inc.) into 96-well microtiter plates at about 1×10⁴ cells per well and cultured overnight. The cells are then washed in inositol-free DMEM and incubated about 20 hr in inositol-free DMEM with 0.3% bovine serum albumin supplemented with 0.80 μCi/well of myo-1,2⁻³H-inositol to allow for the uptake of the [³H]-myo-inositol and incorporation into phosphatidyl inositol. The inositol containing medium is removed and the cells are washed twice with assay buffer (MEM containing 10 mM LiCl, 20 mM HEPES, and 1 mg/ml BSA). The cells are then incubated for 20 min at 37° C. in assay buffer. Various concentrations of potential agonists are added to the wells for about 1 hr. At the end of this period, the medium is removed and the cells are lysed and analyzed as in Example 3.

### EXAMPLE 8

Demonstration of the Invention in Wild-Type HEK293 Cells Naturally Expressing Acetylcholine Receptors

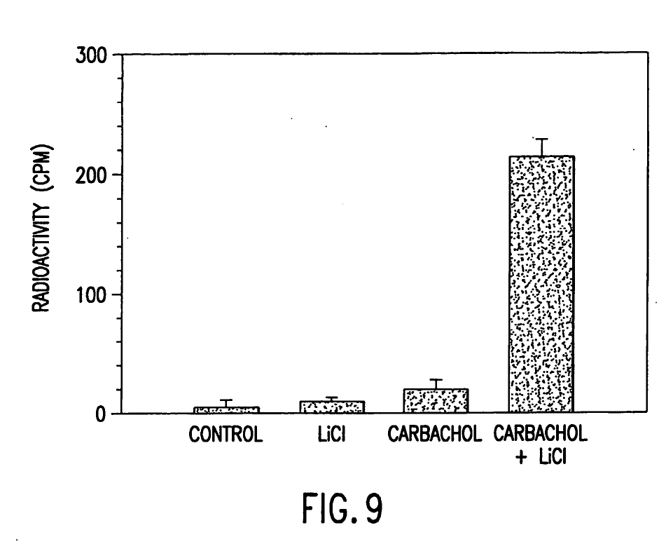

Human embryonic kidney (HEK293) cells were obtained from ATCC and were cultured in DMEM Glutamax (Gibco BRL) containing 10% fetal bovine serum, 100 U/ml penicillin and 100 μg/ml streptomycin. Cells were treated as described in Example 3 for M1-CHO cells. Final concentrations of LiCl and carbachol were 5 mM and 1 mM where used. Data from the HEK cells are shown in FIG. 9. It can be seen that the assay detected the increase of inositol phosphate caused by activation of acetylcholine receptors by carbachol. This shows that the present invention can be used in cells such as these HEK293 cells that naturally express a GPCR for which it is desired to identify agonists.

### EXAMPLE 9

Demonstration of the Invention in T24 Cells Stably Expressing the Human M1 Muscarinic Acetylcholine Receptor

T24 cells were obtained from ATCC and were cultured in DMEM Glutamax (Gibco BRL) containing 10% fetal bovine serum, 100 U/ml penicillin and 100 μg/ml streptomycin. The human muscarinic M1 receptor cDNA (GenBank accession no. M35128) was amplified from a human cDNA library using PCR and cloned into the EcoRV/BamH1 sites of pIRES/Neo (Invitrogen; GenBank accession no. U89673) by scientists at the Banyu Tsukuba Research Institute. This construct was obtained from Banyu and transfected into T24 cells using Lipofectamine 2000 (Gibco BRL). Stably transfected clones were selected by growth in medium containing 0.4 mg/ml Geneticin (Gibco BRL). It was found the parent cell line did not express muscarinic receptors. This observation was based on there being no rise in intracellular calcium upon treatment of cells with carbachol in the Molecular Devices FLIPR system using the manufacturer's recommended protocols for intracellular calcium. Clones expressing functional muscarinic receptors were identified on the basis of a robust increase in intracellular calcium following treatment with carbachol as observed with the M1-CHO cell line described above.

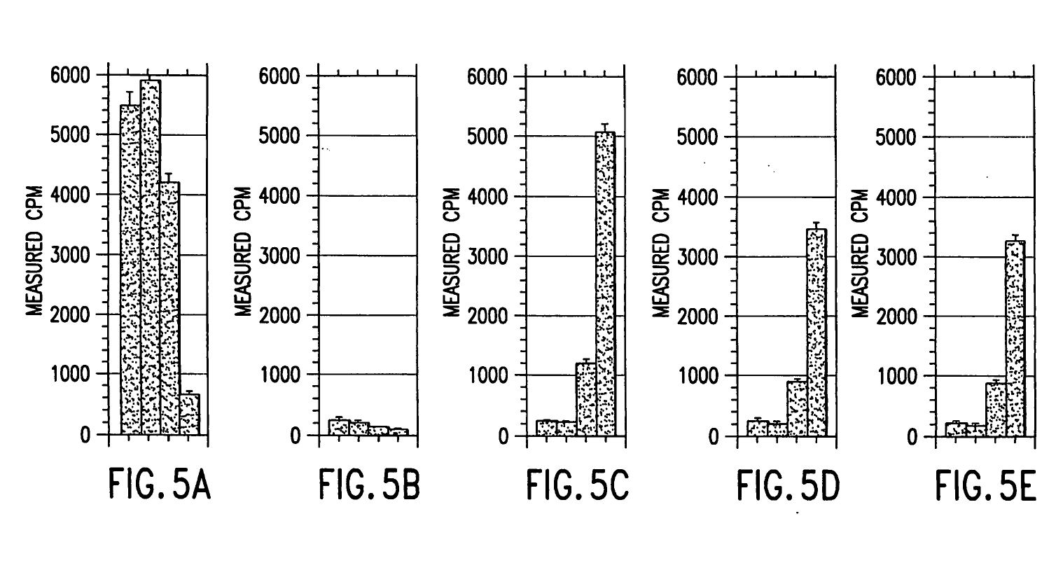

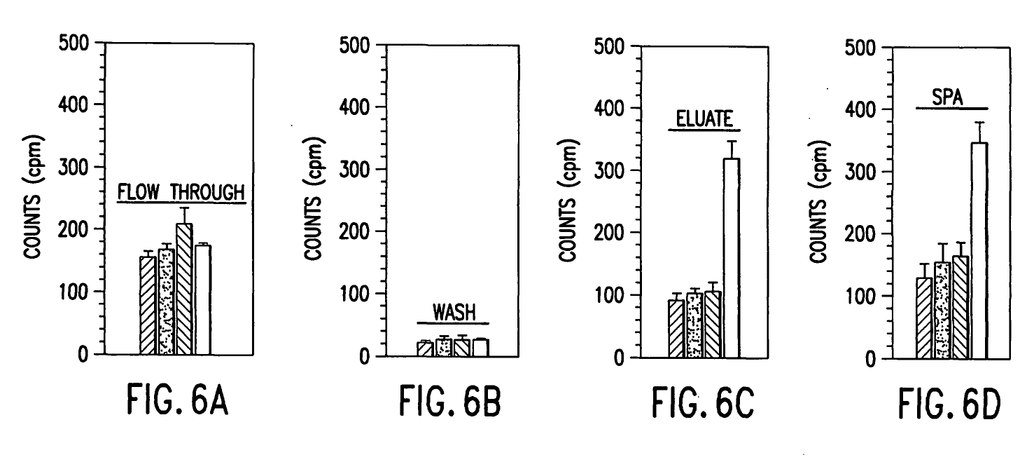

Data from one clonal M1-T24 cell line are shown to demonstrate the invention (see FIG. 5). Cells were treated as described in Example 3 for M1-CHO cells except that lysis was accomplished by incubating cells with 200 μl/well of 0.2M formic acid for 20 minutes at room temperature rather than the freeze and thaw cycle described for M1-CHO cells. Final concentrations of LiCl and carbachol were 5 mM and 1 mM where used. There was no response to carbachol in untransfected T24 cells.

### EXAMPLE 10

Demonstration of the Invention in CHO/NFAT Stably Expressing the Neuropeptide FF Receptor

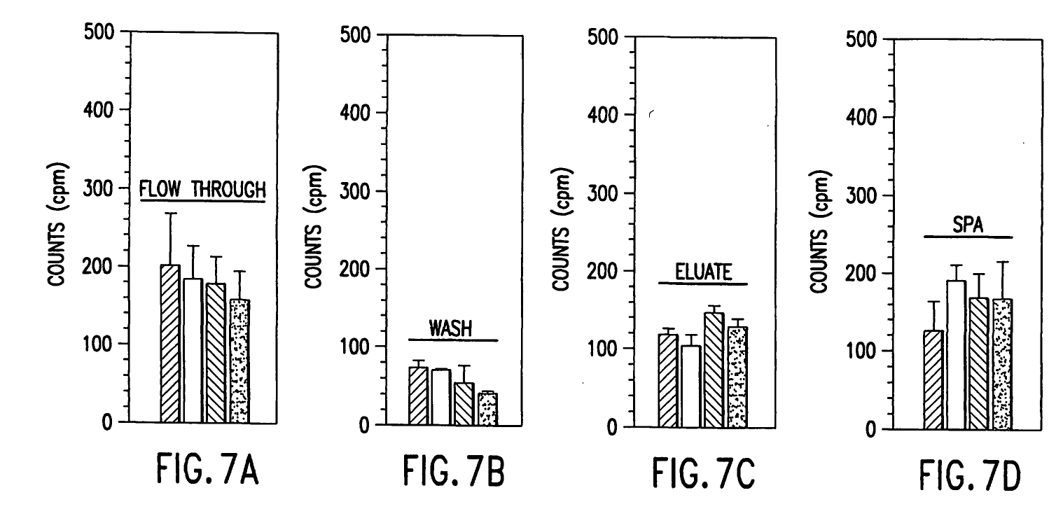

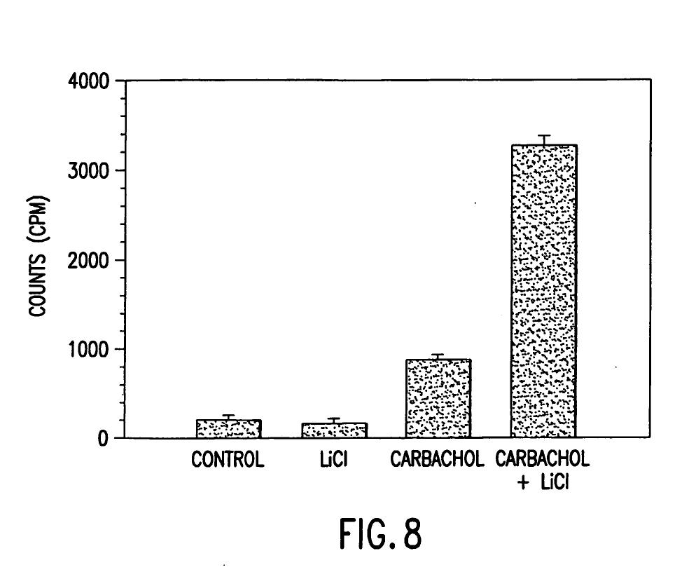

There was no response to NPFF in untransfected CHO/NFAT cells (FIGS. 7A-D).

### EXAMPLE 11

Demonstration of the Invention Using Poly-L-Lysine Beads

### EXAMPLE 12

Plating of Cells for Example 3

Standard Reagents

0.2 M carbachol

Sigma C4382, lot 79H0110, 365 mg/10 mL—aliquoted and stored at −20° C.

2 M LiCl

Sigma L4408, lot 108H02031, 4.24 g/50 mL—aliquoted and stored at −20° C.

1 M CaCl₂

Sigma C3881, Lot 79H11144, 7.35 g/50 mL—aliquoted and stored at −20° C.

myo-inositol

Sigma I5125, lot 49H0390, not sufficiently soluble to make a stock solution—dissolve directly in buffer, Mr 180.2,40 mM=7.2 mg/mL

scyllo-inositol

Sigma I8132, lot 97H11118, not sufficiently soluble to make a stock solution—dissolve directly in buffer, Mr 180.2, 40 mM=7.2 mg/mL

Eluo3-AM

Mol. Probes F-1241, lot 2801-1 Mr 1129.86. Dissolve 1 mg in 443 μL DMSO, split to 2×220 μL aliquots—2 mM stock—store at −80° C.

Thrombin

Lot HT1360A from Enzyme Research Laboratories. 91.5 μM stock stored in 5 μL aliquots at −80° C. Dilute 2.2 μL to 5 mL—final 40 nM

Hanks' BSS

Gibco BRL 14175-079, lot 1064139

MEM

Gibco BRL 41090-036, lot 1067713

F-12

Gibco BRL 31765-035, lot 1061014

Pluronic F-127

Mol. Probes P-3000, lot 0111-62

FBS

Hyclone SH30071.01, lot AGK7211—dispensed into 10 mL aliquots—stored at −20° C.

DMSO

BSA

Sigma A9647, lot 39H1111

Probenecid

Sigma P8761, lot 129H0972

1 M Hepes

Gibco BRL 15630-080, lot 1066035

4-bromo-A-23187

Mol. Probes B-1494, lot 1001-3, Mr 602.52. dissolve 1 mg in 166 μL DMSO to 10 mM; final 10 μM, stored as 25 μL aliquots at −80° C. Dilute 20 μL to 5 mL—final 40 μM

Trypsin/EDTA

Gibco BRL 26300-054, lot 1067154

Antibiotics (pen/strep)

Gibco BRL 15140-122, lot 1063021

Working Solutions (Prepare Daily for 4 Plates Worth)

Fluo-3 AM dye stock (keep in dark)

Mix 210 μL 2 mM stock with 210 μL Pluronic F-127 solution.

Probenecid

Dissolve 710 mg in 3 mL 1 M NaOH.

Assay Buffer (Final pH 7.0)—make 2 L

| | | |
|---|---|---|
| 1 | L | Hanks' BSS |
| 20 | mL | 1 M Hepes |
| 2 | mL | 1 M CaCl₂ |
| 1 | g | BSA |
| 3 | mL | Probenecid (add last; final 2.5 mM) |

Dye Loading Buffer (Keep in Dark)

| | |
|---|---|
| 49 mL | Assay buffer |
| 0.5 mL | FBS |
| 0.4 mL | Fluo-3 dye stock |

Plating Cells

M1-CHO: Harvest cells from 2×T150 into 20 mL MEM/10% FCS/Pen/strep. Count and dilute to 0.8×10⁶/mL. Plate 100 μL/well in 4×96-well black plates. Do 24 hr before experiment. Use 25-250 μL biohit multichannel pipettor set on rP mode. Up speed 2, down speed 1, touch to sides of wells when dispensing. Pipette up/down twice before going to plate. After dispensing, tap the plate to even the distribution of cell suspension in the well.

Cell Washer

Cell washer is now calibrated to leave 100 μL in each well with Costar plates. Settings are 802F to wash from growth medium into assay buffer, 8010 to drain to 100 μL, and 804F for wash after dye loading. These wash speeds/heights leave the cells on the plate undisturbed but should still be vigorous enough to wash properly. Hold wash buffer at 37° C.

Dye Loading

Wash cells on 802F with buffer and drain (8010) to leave 100 μL/well. Add 100 μL dye loading buffer/well. Incubate at 37° C. for 1 hr.

Experiment Environment Conditions

35° C. as per FLIPR factory settings.

Materials

Assay plates—Costar 3603-96 well black w/clear bottom, sterile tissue culture treated

Robot tips—

Reagent reservoirs—sterile: Labcor 730-004 (Fisher xx-xxx-xx)

non-sterile: Labcor 730-001 (Fisher 13-681-100)

Addition plates—Costar 3363-96 well V-bottom clear polpropylene non-sterile

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of which are incorporated by reference in their entireties.

What is claimed is:

1. A method of measuring inositol phosphates in cells that comprises:
(a) preparing a lysate from cells in which inositol phosphates have been radiolabeled;
(b) mixing the lysate with a solid phase that is a material that contains positive charges on its surface and a scintillant within so that the radiolabeled inositol phosphates in the lysate adhere to the solid phase and activate the scintillant; and
(c) measuring the amount of scintillation from the solid phase.

2. A method of identifying inhibitors of an inositol phosphate phosphatase comprising:
(a) adding inositol that has been labeled with a radioisotope to test cells that express the inositol phosphate phosphatase so that the inositol that has been labeled with a radioisotope is incorporated into inositol phosphates in the test cells;
(b) incubating the test cells with a substance for a period sufficient for the substance to inhibit inositol phosphate phosphatases in the test cells;
(c) lysing the test cells and preparing a test lysate from the test cells;
(d) bringing the test lysate into contact with a solid phase so that inositol phosphates from the test lysate adhere to the solid phase while inositol from the test lysate does not adhere to the solid phase;
(e) determining the amount of radioactivity adhered to the solid phase in step (d);
(f) adding inositol that has been labeled with a radioisotope to control cells that express the inositol phosphate phosphatase so that the inositol that has been labeled with a radioisotope is incorporated into inositol phosphates in the control cells;
(g) incubating the control cells in the absence of the substance for a period essentially the same as the period in step (b);
(h) lysing the control cells and preparing a control lysate from the control cells;
(i) bringing the control lysate into contact with a solid phase so that inositol phosphates from the control lysate adhere to the solid phase while inositol from the control lysate does not adhere to the solid phase;
(j) determining the amount of radioactivity adhered to the solid phase in step (i);
where if the amount of radioactivity determined in step (e) is greater than the amount of radioactivity determined in step (j) then the substance is an inhibitor of the inositol phosphate phosphatase.

3. The method of claim 2 where adding inositol that has been labeled with a radioisotope to the test cells and the control cells is done by growing or incubating the test cells and control cells in inositol-free medium and then adding radiolabeled inositol to the medium or changing the medium to a medium that contains radiolabeled inositol.

4. The method of claim 3 where the test cells and control cells are grown or incubated for about 4 to 40 hr in the presence of the inositol that has been labeled with a radioisotope.

5. The method of claim 2 where the inositol that is added to the test cells and control cells is labeled with ³H or ¹⁴C.

6. The method of claim 2 where the test cells and control cells are present in the wells of a multiwell microtiter plate.

7. The method of claim 2 where the incubations of steps (b) and (g) are carried out for a period of from 30 seconds to 24 hr.

8. The method of claim 2 where the solid phase is a glass bead doped with Ce, Mn, Cu, Pb, Sn, Au, Ag, or Sm.

9. The method of claim 2 where the solid phase is yttrium silicate doped with Ce (Y₂SiO₅:Ce) formed into beads.

10. The method of claim 2 where the test cells and control cells are selected from the group consisting of: L cells L-M(TK⁻) (ATCC CCL 1.3), L cells L-M (ATCC CCL 1.2), HEK293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), T24 (ATCC HTB4), and MRC-5 (ATCC CCL 171).

11. The method of claim 2 where the solid phase is a multiwell tissue culture plate in which the walls and/or the bottoms of the wells have been impregnated with a scintillant.

12. A method of identifying inhibitors of an inositol phosphate phosphatase comprising:
(a) adding [³H]-myo-inositol to mammalian test cells that express the inositol phosphate phosphatase so that the [³H]-myo-inositol is incorporated into inositol phosphates in the test cells;
(b) incubating the test cells with a substance for a period sufficient for the substance to inhibit inositol phosphate phosphatases in the test cells;
(c) lysing the test cells and preparing a test lysate from the test cells;
(d) bringing the test lysate into contact with a solid phase that is yttrium silicate doped with Ce (Y₂SiO₅:Ce) formed into beads so that inositol phosphates from the test lysate adhere to the solid phase while inositol from the test lysate does not adhere to the solid phase;
(e) determining the amount of radioactivity adhered to the solid phase in step (d) by mixing the solid phase in step (d) with scintillation fluid and counting in a scintillation counter;
(f) adding [³H]-myo-inositol to mammalian control cells that express the inositol phosphate phosphatase so that the [³H]-myo-inositol is incorporated into inositol phosphates in the control cells;
(g) incubating the control cells in the absence of the substance for a period essentially the same as the period in step (b);
(h) lysing the control cells and preparing a control lysate from the control cells;
(i) bringing the control lysate into contact with a solid phase that is yttrium silicate doped with Ce (Y₂SiO₅:Ce) formed into beads so that inositol phosphates from the control lysate adhere to the solid phase while inositol from the control lysate does not adhere to the solid phase;
(j) determining the amount of radioactivity adhered to the solid phase in step (i) by mixing the solid phase in step (i) with scintillation fluid and counting in a scintillation counter;
where if the amount of radioactivity determined in step (e) is greater than the amount of radioactivity determined in step (i) then the substance is an inhibitor of the inositol phosphate phosphatase.

13. A method of identifying agonists of a G-protein coupled receptor (GPCR) comprising:
(a) adding inositol that has been labeled with a radioisotope to test cells expressing the GPCR so that the inositol that has been labeled with a radioisotope is incorporated into inositol and inositol phosphates in the test cells;
(b) incubating the test cells with a substance for a period sufficient for the substance to activate the GPCR in the test cells;
(c) lysing the test cells and preparing a test lysate from the test cells;
(d) adding the test lysate to a solid phase so that inositol phosphates from the test lysate adhere to the solid phase while inositol from the test lysate does not adhere to the solid phase;
(e) determining the amount of radioactivity adhered to the solid phase in step (d);
(f) adding inositol that has been labeled with a radioisotope to control cells expressing the GPCR so that the inositol that has been labeled with a radioisotope is incorporated into inositol and inositol phosphates in the control cells;
(g) incubating the control cells in the absence of the substance for a period essentially the same as the period in step (b);
(h) lysing the control cells and preparing a control lysate from the control cells;
(i) adding the control lysate to a solid phase so that inositol phosphates from the control lysate adhere to the solid phase while inositol from the control lysate does not adhere to the solid phase;
(j) determining the amount of radioactivity adhered to solid phase in step (i);
where if the amount of radioactivity determined in step (e) is greater than the amount of radioactivity determined in step (j) then the substance is an agonist of the GPCR.

14. The method of claim 13 where LiCl to a final concentration of about 0.5 mM to 10 mM is present in steps (b) and (g).

15. The method of claim 13 where adding inositol that has been labeled with a radioisotope to the test cells and the control cells is done by growing or incubating the test cells and control cells in inositol-free medium and then adding radiolabeled inositol to the medium or changing the medium to a medium that contains radiolabeled inositol.

16. The method of claim 13 where the test cells and control cells are grown or incubated for about 4 to 40 hr in the presence of the inositol that has been labeled with a radioisotope.

17. The method of claim 13 where the inositol that is added to the test cells and control cells is labeled with ³H or ¹⁴C.

18. The method of claim 13 where the test cells and control cells are present in the wells of a multiwell microtiter plate.

19. The method of claim 13 where the incubations of steps (b) and (g) are carried out for a period of from 30 seconds to 24 hr.

20. The method of claim 13 where the solid phase is a glass bead doped with Ce, Mn, Cu, Pb, Sn, Au, Ag, or Sm.

21. The method of claim 13 where the solid phase is yttrium silicate doped with Ce (Y₂SiO₅:Ce) formed into beads.

22. The method of claim 13 where the test cells and control cells are selected from the group consisting of: L cells L-M(TK⁻) (ATCC CCL 1.3), L cells L-M (ATCC CCL 1.2), HEK293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), T24 (ATCC HTB4), and MRC-5 (ATCC CCL 171).

23. The method of claim 13 where the test cells and control cells naturally express the GPCR.

24. The method of claim 13 where the test cells and control cells do not naturally express the GPCR but have been transfected with an expression vector encoding the GPCR so that the GPCR is expressed in the test cells and control cells.

25. The method of claim 13 where the GPCR is selected from the group consisting of: human M1 muscarinic acetylcholine receptor, human neuropeptide FF receptor, human luteinizing hormone releasing hormone receptor, human neurokinin 1 receptor, human neurokinin 3 receptor, human chemokine receptor CCR2b, human substance P receptor, human neuromedin K receptor, human metabotropic glutamate receptor 1 alpha, human thrombin receptor, human M5 muscarinic acetylcholine receptor, rat M5 muscarinic acetylcholine receptor, and the human histamine receptor.

26. The method of claim 13 where the solid phase is a multiwell tissue culture plate in which the walls and/or the bottoms of the wells have been impregnated with a scintillant.

27. A method of identifying agonists of a G-protein coupled receptor (GPCR) comprising:
(a) adding inositol that has been labeled with a radioisotope to test cells expressing the GPCR so that the inositol that has been labeled with a radioisotope is incorporated into inositol phosphates in the test cells;
(b) incubating the test cells with a substance for a period sufficient for the substance to activate the GPCR in the test cells;
(c) lysing the test cells and preparing a test lysate from the test cells;
(d) adding the test lysate to a solid phase so that inositol phosphates from the test lysate adhere to the solid phase while inositol from the test lysate does not adhere to the solid phase;
(e) determining the amount of radioactivity adhered to solid phase in step (d);
(f) adding inositol that has been labeled with a radioisotope to control cells that are substantially identical to the test cells except that the control cells do not express the GPCR so that the inositol that has been labeled with a radioisotope is incorporated into inositol and inositol phosphates in the control cells;
(g) incubating the control cells with the substance for a period essentially the same as the period in step (b);
(h) lysing the control cells and preparing a control lysate from the control cells;
(i) adding the control lysate to a solid phase so that inositol phosphates from the control lysate adhere to the solid phase while inositol from the control lysate does not adhere to the solid phase;
(j) determining the amount of radioactivity adhered to the solid phase in step (i);
where if the amount of radioactivity determined in step (e) is greater than the amount of radioactivity determined in step (j) then the substance is an agonist of the GPCR.

28. The method of claim 27 where LiCI to a final concentration of about 0.5 mM to 10 mM is present in steps (b) and (g).

29. The method of claim 27 where adding inositol that has been labeled with a radioisotope to the test cells and the control cells is done by growing or incubating the test cells and control cells in inositol-free medium and then adding radiolabeled inositol to the medium or changing the medium to a medium that contains radiolabeled inositol.

30. The method of claim 27 where the test cells and control cells are grown or incubated for about 4 to 40 hr in the presence of the inositol that has been labeled with a radioisotope.

31. The method of claim 27 where the inositol that is added to the test cells and control cells is labeled with ³H or ¹⁴C.

32. The method of claim 27 where the test cells and control cells are present in the wells of a multiwell microtiter plate.

33. The method of claim 27 where the incubations of steps (b) and (g) are carried out for a period of from 30 seconds to 24 hr.

34. The method of claim 27 where the solid phase is a glass bead doped with Ce, Mn, Cu, Pb, Sn, Au, Ag, or Sm.

35. The method of claim 27 where the solid phase is yttrium silicate doped with Ce (Y₂SiO₅:Ce) formed into beads.

36. The method of claim 27 where the test cells and control cells are selected from the group consisting of: L cells L-M(TK⁻) (ATCC CCL 1.3), L cells L-M (ATCC CCL 1.2), HEK293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), T24 (ATCC HTB4), and MRC-5 (ATCC CCL 171).

37. The method of claim 27 where the test cells naturally express the GPCR.

38. The method of claim 27 where the test cells do not naturally express the GPCR but have been transfected with an expression vector encoding the GPCR so that the GPCR is expressed in the test cells.

39. The method of claim 27 where the GPCR is selected from the group consisting of: human M1 muscarinic acetylcholine receptor, human neuropeptide FF receptor, human luteinizing hormone releasing hormone receptor, human neurokinin 1 receptor, human neurokinin 3 receptor, human chemokine receptor CCR2b, human substance P receptor, human neuromedin K receptor, human metabotropic glutamate receptor 1 alpha, human thrombin receptor, human M5 muscarinic acetylcholine receptor, rat M5 muscarinic acetylcholine receptor, and the human histamine receptor.

40. The method of claim 27 where the solid phase is a multiwell tissue culture plate in which the walls and/or the bottoms of the wells have been impregnated with a scintillant.

41. A method of identifying agonists of a G-protein coupled receptor (GPCR) comprising:
(a) adding [³H]-myo-inositol to mammalian test cells expressing the GPCR so that the [³H]-myo-inositol is incorporated into inositol and inositol phosphates in the test cells;
(b) incubating the test cells with a substance for a period sufficient for the substance to activate the GPCR in the test cells;
(c) lysing the test cells and preparing a test lysate from the test cells;
(d) adding the test lysate to a solid phase that is yttrium silicate doped with Ce (Y₂SiO₅:Ce) formed into beads so that inositol phosphates from the test lysate adhere to the solid phase while inositol from the test lysate does not adhere to the solid phase;
(e) determining the amount of radioactivity adhered to the solid phase in step (d) by mixing the solid phase in step (d) with scintillation fluid and counting in a scintillation counter;
(f) adding [³H]-myo-inositol to mammalian control cells expressing the GPCR so that the [³H]-myo-inositol is incorporated into inositol and inositol phosphates in the control cells;
(g) incubating the control cells in the absence of the substance for a period essentially the same as the period in step (b);
(h) lysing the control cells and preparing a control lysate from the control cells;
(i) adding the control lysate to a solid phase that is yttrium silicate doped with Ce (Y₂SiO₅:Ce) formed into beads so that inositol phosphates from the control lysate adhere to the solid phase while inositol from the control lysate does not adhere to the solid phase;
(j) determining the amount of radioactivity adhered to the solid phase in step (i) by mixing the solid phase in step (i) with scintillation fluid and counting in a scintillation counter;
where if the amount of radioactivity determined in step (e) is greater than the amount of radioactivity determined in step (j) then the substance is an agonist of the GPCR.

42. A method of identifying agonists of a G-protein coupled receptor (GPCR) comprising:
(a) adding [³H]-myo-inositol to mammalian test cells expressing the GPCR so that the [³H]-myo-inositol is incorporated into inositol phosphates in the test cells;
(b) incubating the test cells with a substance for a period sufficient for the substance to activate the GPCR in the test cells;
(c) lysing the test cells and preparing a test lysate from the test cells;
(d) adding the test lysate to a solid phase that is yttrium silicate doped with Ce (Y₂SiO₅:Ce) formed into beads so that inositol phosphates from the test lysate adhere to the solid phase while inositol from the test lysate does not adhere to the solid phase;
(e) determining the amount of radioactivity adhered to solid phase in step (d) by mixing the solid phase in step (d) with scintillation fluid and counting in a scintillation counter;
(f) adding [³H]-myo-inositol to mammalian control cells that are substantially identical to the test cells except that the control cells do not express the GPCR so that the [³H]-myo-inositol is incorporated into inositol and inositol phosphates in the control cells;
(g) incubating the control cells in the absence of the substance for a period essentially the same as the period in step (b);
(h) lysing the control cells and preparing a control lysate from the control cells;
(i) adding the control lysate to a solid phase that is yttrium silicate doped with Ce (Y₂SiO₅:Ce) formed into beads so that inositol phosphates from the control lysate adhere to the solid phase while inositol from the control lysate does not adhere to the solid phase;
(j) determining the amount of radioactivity adhered to solid phase in step (i) by mixing the solid phase in step (i) with scintillation fluid and counting in a scintillation counter;
where if the amount of radioactivity determined in step (e) is greater than the amount of radioactivity determined in step (j) then the substance is an agonist of the GPCR.

43. A method of identifying antagonists of a G-protein coupled receptor (GPCR) comprising:
(a) adding inositol that has been labeled with a radioisotope to test cells expressing the GPCR so that the inositol that has been labeled with a radioisotope is incorporated into inositol and inositol phosphates in the test cells;
(b) incubating the test cells with a known agonist of the GPCR and a substance for a period sufficient for the agonist to activate the GPCR in the test cells if the substance is not an antagonist;
(c) lysing the test cells and preparing a test lysate from the test cells;
(d) adding the test lysate to a solid phase so that inositol phosphates from the test lysate adhere to the solid phase while inositol from the test lysate does not adhere to the solid phase;
(e) determining the amount of radioactivity adhered to the solid phase in step (d);
(f) adding inositol that has been labeled with a radioisotope to control cells expressing the GPCR so that the inositol that has been labeled with a radioisotope is incorporated into inositol and inositol phosphates in the control cells;
(g) incubating the control cells in the presence of the agonist but in the absence of the substance for a period essentially the same as the period in step (b);
(h) lysing the control cells and preparing a control lysate from the control cells;
(i) adding the control lysate to a solid phase so that inositol phosphates from the control lysate adhere to the solid phase while inositol from the control lysate does not adhere to the solid phase;
(j) determining the amount of radioactivity adhered to the solid phase in step (i);
where if the amount of radioactivity determined in step (i) is greater than the amount of radioactivity determined in step (e) then the substance is an agonist of the GPCR.
